(19) United States
(12) Patent Application Publication
(10) Pub. No.: US 2013/0158072 A1
(43) Pub. Date: Jun. 20, 2013

(54) KAPPA OPIOID RECEPTOR BINDING LIGANDS

(71) Applicants: Frank Ivy Carroll, Durham, NC (US); James B. Thomas, Efland, NC (US); S. Wayne Mascarella, Hillsborough, NC (US)

(72) Inventors: Frank Ivy Carroll, Durham, NC (US); James B. Thomas, Efland, NC (US); S. Wayne Mascarella, Hillsborough, NC (US)

(73) Assignee: Research Triangle Institute, Research Triangle Park, NC (US)

(21) Appl. No.: 13/518,158

(22) PCT Filed: Jan. 19, 2010
(86) PCT No.: PCT/US2010/021385

§ 371 Date: Sep. 20, 2012

(51) Int. Cl. A61K 31/4725; A61K 45/06; C07D 401/12

(52) U.S. Cl. CPC A61K 31/4725; C07D 401/12; A61K 45/06
USPC 514/307; 546/146

(57) ABSTRACT

Kappa opioid recep-tor antagonists are provided that yield significant improvements in functional binding assays to kappa opioid receptors, and the use of these antagonists in treatment of disease states that are ameliorated by binding of the kappa opioid receptor, such as heroin or cocaine addictions.

### BACKGROUND OF THE INVENTION

1. Field of Invention

The present invention relates to compounds that bind with high affinity and/or specificity to kappa opioid receptors.

2. Discussion of the Background

Stress can induce despair and increase the risk of clinical depression and drug abuse.^{1,2} Dynorphin, the endogenous ligand for the κ-opioid receptor, is a stress-related neuropeptide in the brain that may mediate these responses.³ Activation of the κ-opioid receptor causes place aversion in rodents and dysphoria in humans.^{4,5} The dynorphin/κ-opioid receptor system has been reported to be critical for stress-induced depression-like behaviors and reinstatement to drug seeking behavior.^{4,6-10} The results from these studies have led to an increased interest in selective κ-opioid receptor antagonists.

The first non-peptide, highly selective antagonists of the κ-opioid receptor were nor-BNI¹¹ (1, FIG. 1) and GNTI¹² (2, FIG. 1), which were derived from the non-selective opioid receptor antagonist naltrexone. More recently, JDTic (3, FIG. 1) was discovered as the first highly potent and selective κ-opioid receptor antagonist from the N-substituted trans-3,4-dimethyl-4-(3-hydroxyphenyl)piperidine (4, FIG. 1) class of antagonist,^{13,14} and arodyn (5, FIG. 1) was developed from dynorphin.¹⁵ Studies with these compounds have shown that this system is intimately involved in brain processes that relate to stress, fear, and anxiety as well as reward-seeking behavior.¹⁶ Studies have shown that 3 and 1 dose-dependently reduce fear and stress-induced responses in multiple behavioral paradigms with rodents (immobility in the forced-swim assay,^{8,10} reduction of exploratory behavior in the elevated plus maze, fear-potentiated startle). Furthermore, selective κ antagonists have been shown to reduce stress-induced reinstatement of cocaine self-administration in rats,⁸ block the stress-induced potentiation of cocaine place preference conditioning,^{7,9,18} decrease dependence-induced ethanol self-administration,¹⁹ diminish deprivation-induced eating in rats,²⁰ and prevent pre-pulse inhibition mediated by the κ agonist U50,488.²¹ These observations regarding the behavioral consequences of receptor blockade in several animal tests suggest that κ antagonists might be useful for the treatment of anxiety, depression, schizophrenia, addiction, and eating disorders.

In vivo, 3 has been shown to be more potent at blocking κ-opioid agonist-induced activity than other κ-opioid antagonist.²² Compound 3 was also shown to have oral activity in antagonizing the antinociceptive activity of the κ agonist enadoline in mice²² and preventing stress-induced cocaine reinstatement of self-administration in rats.⁸ To the present Inventors' knowledge, 3 remains the only orally active κ-opioid receptor antagonist.

In a recent structure activity relationship study, it was reported that 8a (see Table 1), which has an extra methyl group on the (1S)-isopropyl group of 3, had a Kₑ value of 0.03 nM at the κ-opioid receptor, relative to 0.02 nM for 3, and retained 100- and 800-fold κ selectivity relative to the μ and δ, opioid receptors, respectively.²³ It was also reported that the methyl ether 8b (see Table 1) was a highly potent antagonist with a Kₑ value of 0.06 nM at the κ-opioid receptor, making it only 3-fold less potent than 3. Compound 8b was 857- and 1970-fold selective for the κ receptor relative to the μ and δ receptors, respectively. The synthesis of the N-methyl analogue 8c has also been reported; however, this analogue had not been evaluated for inhibition of agonist-stimulated [³⁵S]GTPγS binding at cloned μ-, δ-, and κ-opioid receptors in the Inventors' laboratory.¹⁴

The present invention described the synthesis of a series of analogues of 3 (see Table 1 exemplary structures) and report results on their ability to inhibit agonist-stimulated [³⁵S]GTPγS binding in cells expressing cloned 15-, and κ-opioid receptors. Even though 3 has drug-like properties and has performed well in several animal behavioral tests,^{8,17,22} analogues thereof may have better pharmacokinetic properties and ability to penetrate the brain. All of the analogues described herein had calculated log BB values²⁴ that suggested they would possess better brain penetration than 3. All the mono- and di-methylated 3 analogues with the exception of 8k had subnanomolar Kₑ values at the κ-opioid receptor. Analogues 8d and previously reported 8a and 8b were are potent and selective κ antagonists.

Additional reports of the earlier work in the class of compounds is described in, for example, U.S. patent publication No. 2002/0132828, U.S. Pat. No. 6,974,824, U.S. patent publication No. 2006/0183743, and U.S. patent publication No. 2009/0264462.

### SUMMARY OF THE INVENTION

It is an object of the invention to provide compounds which bind to kappa opioid receptors with high affinity, in particular phenoxy ether derivatives of compound 3.

It is another object of the invention to provide compounds which bind to kappa opioid receptors with high specificity.

It is another object of the invention to provide compounds which bind to kappa opioid receptors with high affinity and specificity in functional assays.

The objects of the present invention, and others, are accomplished with the compounds, compositions and methods described below which have the above advantages described above.

### BRIEF DESCRIPTION OF THE FIGURES

A more complete appreciation of the invention and many of the attendant advantages thereof will be readily obtained as the same becomes better understood by reference to the following detailed description when considered in connection with the accompanying drawings, wherein:

FIG. 1: chemical structure of compounds 1-5.

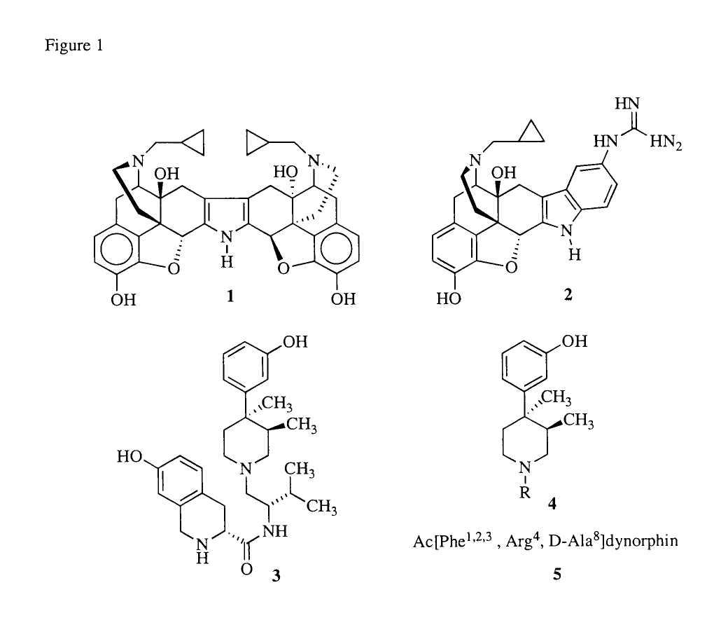

FIG. 2: examples of synthetic routes to compounds of formula 8.

FIG. 3: synthesis of intermediates.

FIG. 4: synthesis of intermediates.

### DETAILED DESCRIPTION OF THE INVENTION

The present invention provides kappa opioid antagonists that bind to kappa opioid receptors with high affinity and/or specificity. Compounds of the present invention are those represented by the formula (I):

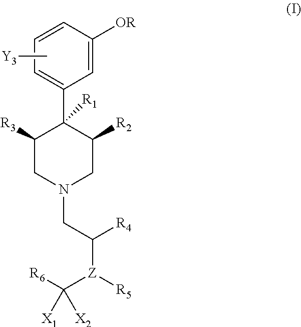

where

R is C₁₋₈ alkyl, C₁₋₈ haloalkyl, C₃₋₈ alkenyl, C₃₋₈ alkynyl or CH₂-aryl substituted by one or more groups Y₁;

R₁ is one of the following structures:

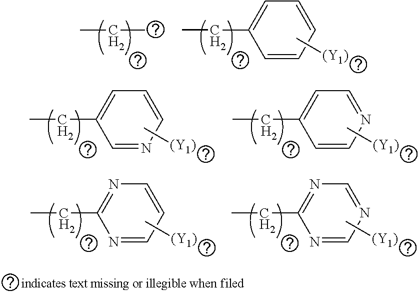

Y₁ is H, OH, Br, Cl, F, CN, CF₃, NO₂, N₃, OR₈, CO₂R₉, C₁₋₆ alkyl, NR₁₀R₁₁, NHCOR₁₂, NHCO₂R₁₂, CONR₁₃R₁₄, or CH₂(CH₂)ₙY₂;

Y₂ is H, CF₃, CO₂R₉, C₁₋₆ alkyl, NR₁₀R₁₁, NHCOR₁₂, NHCO₂R₁₂, CONR₁₃R₁₄, CH₂OH, CH₂OR₈, or COCH₂R₉;

Y₃ is H, OH, Br, Cl, F, CN, CF₃, NO₂, N₃, OR₈, CO₂R₉, C₁₋₆ alkyl, NR₁₀R₁₁, NHCOR₁₂, NHCO₂R₁₂, CONR₁₃R₁₄, or CH₂(CH₂)ₙY₂;

R₂ is H, C₁₋₈ alkyl, C₃₋₈ alkenyl, C₃₋₈ alkynyl or CH₂-aryl substituted by one or more groups Y₁;

R₃ is H, C₁₋₈ alkyl, C₃₋₈ alkenyl, C₃₋₈ alkynyl or CH₂-aryl substituted by one or more groups Y₁,

wherein R₂ and R₃ may be bonded together to form a C₂₋₈ alkyl group;

R₄ is hydrogen, C₁₋₈ alkyl, CO₂C₁₋₈ alkylaryl substituted by one or more groups Y₁, CH₂-aryl substituted by one or more groups Y₁ or CO₂C₁₋₈ alkyl;

Z is N, O or S, wherein when Z is O or S, there is no R₅;

R₅ is H, C₁₋₈ alkyl, C₃₋₈ alkenyl, C₃₋₈ alkynyl, CH₂CO₂C₁₋₈ alkyl, CO₂C₁₋₈ alkyl or CH₂-aryl substituted by one or more groups Y₁;

n is 0, 1, 2 or 3;

o is 0, 1, 2 or 3;

R₆ is a group selected from the group consisting of structures (a)-(p):

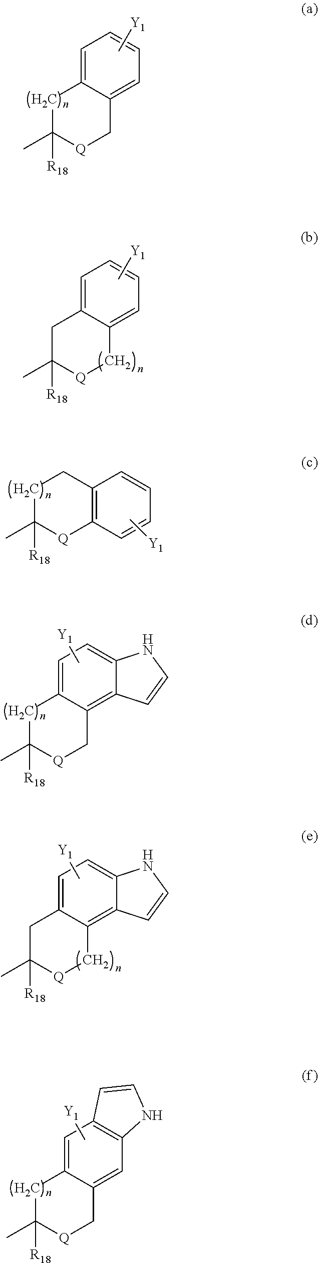

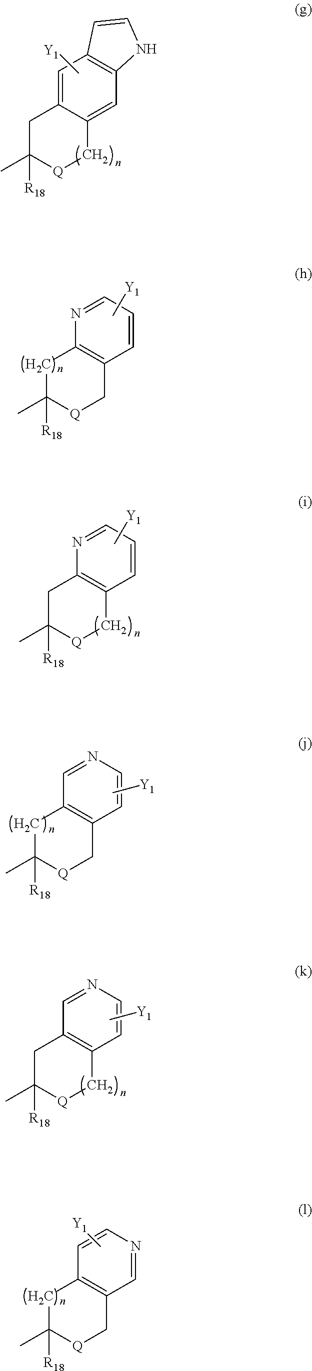

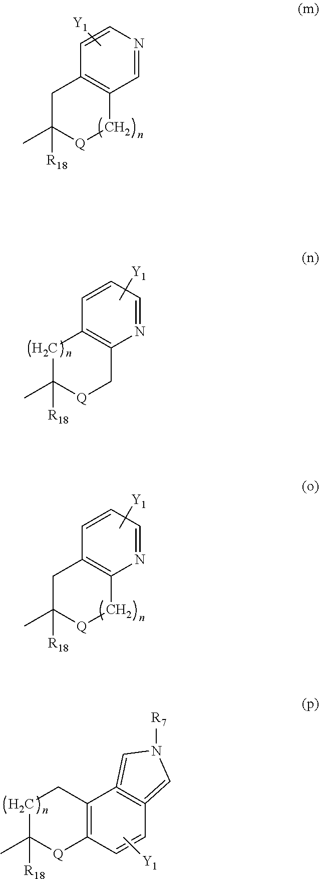

Q is NR₇, CH₂, O, S, SO, or SO₂;

X₁ is hydrogen, C₁₋₈ alkyl, C₃₋₈ alkenyl, or C₃₋₈ alkynyl;

X₂ is hydrogen, C₁₋₈alkyl, C₃₋₈ alkenyl, or C₃₋₈ alkynyl; or X₁ and X₂ together form ═O, ═S, or ═NH;

each R₇ is, independently, H, C₁₋₈ alkyl, CH₂-aryl substituted by one or more substituents Y₁, NR₁₀R₁₁, NHCOR₁₂, NHCO₂R₁₃, CONR₁₄R₁₅, CH₂(CH₂)ₙY₂, or C(═NH)NR₁₆R₁₇;

each of R₈, R₉, R₁₀, R₁₁, R₁₂, R₁₃, R₁₄, R₁₅, R₁₆ and R₁₇ is, independently, H, C₁₋₈ alkyl, CH₂-aryl substituted by one or more substituents H, OH, Br, Cl, F, CN, CF₃, NO₂, N₃, C₁₋₆ alkyl, or CH₂(CH₂)ₙY₂′;

Y₂′ is H, CF₃, or C₁₋₆ alkyl;

R₁₈ is hydrogen, C₁₋₈ alkyl, C₂₋₈ alkenyl, C₃₋₈ alkynyl, or CH₂-aryl substituted by one or more groups Y₁;

and pharmaceutically acceptable salts thereof.

In one preferred embodiment of the present invention:
R is C₁₋₈ alkyl or C₁₋₈ haloalkyl, phenyl substituted by one or more groups Y₁ or CH₂-phenyl substituted by one or more groups Y₁;
R₁ is C₁₋₃ alkyl;
Y₃ is H or C₁₋₆ alkyl;
R₂ is H or C₁₋₈ alkyl,
R₃ is H or C₁₋₈ alkyl,
or R₂ and R₃ are bonded together to form a C₂₋₈ alkyl group
R₄ is H or C₁₋₈ alkyl;
Z is N;
X₁ and X₂ together form ═O;
R₆ is represented by the formula (a), (b) or (c); and
R₁₈ is H or C₁₋₈ alkyl,

or a pharmaceutically acceptable salt thereof.

In another preferred embodiment of the present invention:
R is C₁₋₄ alkyl or C₁₋₄ haloalkyl;
R₁ is C₁₋₃ alkyl;
Y₃ is H or C₁₋₄ alkyl;
R₂ is H or C₁₋₄ alkyl,
R₃ is H or C₁₋₄ alkyl; or R₂ and R₃ are bonded together to form a C₂₋₈ alkyl group R₄ is H or C₁₋₆ alkyl;
Z is N;
X₁ and X₂ together form ═O;
R₆ is represented by the formula (a), (b) or (c); and
R₁₈ is H or C₁₋₄ alkyl,

or a pharmaceutically acceptable salt thereof.

In another preferred embodiment of the present invention:
R is C₁₋₂ alkyl or C₁₋₂ haloalkyl;
R₁ is C₁₋₂ alkyl;
Y₃ is H or C₁₋₂ alkyl;
R₂ is H or C₁₋₂ alkyl,
R₃ is H or C₁₋₂ alkyl, or R₂ and R₃ are bonded together to form a C₂₋₈ alkyl group;
R₄ is hydrogen or C₁₋₆ alkyl;
Z is N;
X₁ and X₂ together form ═O;
R₆ is represented by the formula (a), (b) or (c); and
R₁₈ is H or C₁₋₂ alkyl,

or a pharmaceutically acceptable salt thereof.

In another preferred embodiment of the present invention:
R is methyl or trifluoromethyl;
R₁ is methyl;
Y₃ is H or methyl;
R₂ is H or methyl,
R₃ is H or methyl;
R₄ is H or C₁₋₆ alkyl;
Z is N;
X₁ and X₂ together form ═O;
R₆ is represented by the formula (a), (b) or (c); and
R₁₈ is H or methyl,

or a pharmaceutically acceptable salt thereof.

In another preferred embodiment of the present invention:
R is methyl or trifluoromethyl;
R₁ is methyl;
Y₃ is H;
R₂ is methyl,
R₃ is H;
R₄ is H or C₁₋₄ alkyl;
Z is N;
X₁ and X₂ together form ═O;
R₆ is represented by the formula (a), (b) or (c);
R₁₈ is H or C₁₋₂ alkyl,
Q is NR₇;
R₇ is H or C₁₋₈ alkyl;
Y₁ is H, OH or OR₈;
R₈ is C₁₋₈ alkyl; and
n is 0, 1 or 2,

or a pharmaceutically acceptable salt thereof.

In another preferred embodiment of the present invention:
R is methyl or trifluoromethyl;
R₁ is methyl;
Y₃ is H;
R₂ is methyl,
R₃ is H;
R₄ is hydrogen or C₁₋₄ alkyl;
Z is N;
X₁ and X₂ together form ═O;
R₆ is represented by the formula (a), (b) or (c);
R₁₈ is H or methyl,
Q is NR₇;
R₇ is H or C₁₋₄ alkyl;
Y₁ is H, OH or OR₈;
R₈ is C₁₋₄ alkyl; and
n is 0 or 1,

or a pharmaceutically acceptable salt thereof.

In another preferred embodiment of the present invention:
R is methyl or trifluoromethyl;
R₁ is methyl;
Y₃ is H;
R₂ is methyl,
R₃ is H;
R₄ is H or C₁₋₄ alkyl;
Z is N;
X₁ and X₂ together form ═O;
R₆ is represented by the formula (a), (b) or (c);
R₁₈ is H or methyl,
Q is NR₇;
R₇ is H or C₁₋₂ alkyl;
Y₁ is H, OH or OR₈;
R₈ is C₁₋₂ alkyl; and
n is 0 or 1,

or a pharmaceutically acceptable salt thereof.

In another preferred embodiment of the present invention:
R is methyl;
R₁ is methyl;
Y₃ is H;
R₂ is methyl,
R₃ is H;
R₄ is H or C₁₋₄ alkyl;
Z is N;
X₁ and X₂ together form ═O;
R₆ is represented by the formula (a), (b) or (c);
R₁₈ is H or methyl,
Q is NR_{S};
R₇ is H or methyl;
Y₁ is OH or OR₈;
R₈ is methyl; and
n is 0 or 1,

or a pharmaceutically acceptable salt thereof.

In another preferred embodiment of the present invention:
R is trifluoromethyl;
R₁ is methyl;
Y₃ is H;
R₂ is methyl,
R₃ is H;
R₄ is H or C₁₋₄ alkyl;
Z is N;
X₁ and X₂ together form ═O;
R₆ is represented by the formula (a), (b) or (c);
R₁₈ is H or methyl,
Q is NRS;
R₇ is H or methyl;
Y₁ is OH or OR₈;
R₈ is methyl; and
n is 0 or 1,

or a pharmaceutically acceptable salt thereof.

In another preferred embodiment of the present invention, the kappa opioid receptor antagonist is represented by formula 8d, 8f, 8h, 8k, 8l, 8n or 8p shown in Table 1.

The present invention includes any and all combination of the different structural groups defined above, including those combinations not specifically set forth above. In particular, the present invention includes the combination of each R group with any is C₁₋₈ alkyl, C₁₋₈ haloalkyl, C₃₋₈ alkenyl, C₃₋₈ alkynyl, aryl substituted by one or more groups Y₁ or CH₂-aryl substituted by one or more groups Y₁

As used throughout this disclosure, the terms “alkyl group” or “alkyl radical” encompass all structural isomers thereof, such as linear, branched and cyclic alkyl groups and moieties. Unless stated otherwise, all alkyl groups described herein may have 1 to 8 carbon atoms, inclusive of all specific values and subranges therebetween, such as 2, 3, 4, 5, 6, or 7 carbon atoms.

As used throughout this disclosure, the terms “haloalkyl group” or “haloalkyl radical” encompass all structural isomers thereof, such as linear, branched and cyclic groups and moieties. Unless stated otherwise, all haloalkyl groups described herein may have 1 to 8 carbon atoms, inclusive of all specific values and subranges therebetween, such as 2, 3, 4, 5, 6, or 7 carbon atoms. A C₁₋₂ haloalkyl group is particularly preferred. At least one hydrogen atom is replaced by a halogen atom, i.e., fluorine, chlorine, bromine or iodine. In one embodiment, all of the hydrogen atoms are replaced with halogen atoms. Fluorine is preferred. Perfluoroalkyl groups are particularly preferred. Examples of haloalkyl groups include trifluoromethyl (—CF₃) and perfluoroethyl (—CF₂CF₃).

The alkenyl group or alkynyl group may have one or more double or triple bonds, respectively. As will be readily appreciated, when an alkenyl or alkynyl group is bonded to a heteroatom a double or triple bond is not formed with the carbon atom bonded directly to the heteroatom. Unless stated otherwise, all alkenyl and alkynyl groups described herein may have 3 to 8 carbon atoms, inclusive of all specific values and subranges therebetween, such as 4, 5, 6, or 7 carbon atoms. Preferred examples include —CH₂CH═CH₂ and —CH₂CCH.

The aryl group is a hydrocarbon aryl group, such as a phenyl, naphthyl, phenanthryl, anthracenyl group, which may have one or more C₁₋₄ alkyl group substituents.

The compounds of the present invention are opiates which are preferably antagonists that are selective for the kappa receptor. The κ/μ selectivity may be at least 2:1, but is preferably higher, e.g., at least 5:1, 10:1, 25:1, 50:1, 100:1, 200:1 or even 500:1. The κ/δ selectivity may be at least 2:1, but is preferably higher, e.g., at least 5:1, 10:1, 25:1, 50:1, 100:1, 200:1, 250:1, 500:1, 1000:1, 10,000:1, 15,000:1, 20,000:1, 25,000:1 or even 30,000:1. These ranges include all specific ranges and subranges therebetween as well as all combinations of κ/μ and κ/δ selectivity.

The compounds of the present invention may be in the form of a pharmaceutically acceptable salt via protonation of the amines with a suitable acid. The acid may be an inorganic acid or an organic acid. Suitable acids include, for example, hydrochloric, hydroiodic, hydrobromic, sulfuric, phosphoric, citric, acetic, fumaric, tartaric, and formic acids.

The receptor selectivities discussed above are determined based on the binding affinities at the receptors indicated or their selectivity in opioid functional assays.

The compounds of the present invention may be used to bind opioid receptors. Such binding may be accomplished by contacting the receptor with an effective amount of the inventive compound. Of course, such contacting is preferably conducted in an aqueous medium, preferably at physiologically relevant ionic strength, pH, etc.

The inventive compounds may also be used to treat patients having disease states which are ameliorated by binding opioid receptors or in any treatment wherein temporary suppression of the kappa opioid receptor system is desired. Such diseases states include opiate addiction (such as heroin addiction), cocaine, nicotine, or ethanol addiction. The compounds of the present invention may also be used as cytostatic agents, as antimigraine agents, as immunomodulators, as immunosuppressives, as antiarthritic agents, as antiallergic agents, as virucides, to treat diarrhea, as antipsychotics, as antischizophrenics, as antidepressants, as uropathic agents, as antitussives, as antiaddictive agents, as anti-smoking agents, to treat alcoholism, as hypotensive agents, to treat and/or prevent paralysis resulting from traumatic ischemia, general neuroprotection against ischemic trauma, as adjuncts to nerve growth factor treatment of hyperalgesia and nerve grafts, as anti-diuretics, as stimulants, as anti-convulsants, or to treat obesity. Additionally, the present compounds can be used in the treatment of Parkinson's disease as an adjunct to L-dopa for treatment of dyskinesia associated with the L-dopa treatment.

The compounds of the present invention are particularly useful for treating addiction, such as addiction to cocaine, alcohol, methamphetamine, nicotine, heroine, and other drugs of abuse. With respect to nicotine, the compounds of the present invention are also useful in treating nicotine withdrawal effects.

The compounds may be administered in an effective amount by any of the conventional techniques well-established in the medical field. For example, the compounds may be administered orally, intraveneously, or intramuscularly. When so administered, the inventive compounds may be combined with any of the well-known pharmaceutical carriers and additives that are customarily used in such pharmaceutical compositions. For a discussion of dosing forms, carriers, additives, pharmacodynamics, etc., see Kirk-Othmer Encyclopedia of Chemical Technology, Fourth Edition, Vol. 18, 1996, pp. 480-590, incorporated herein by reference. The patient is preferably a mammal, with human patients especially preferred. Effective amounts are readily determined by those of ordinary skill in the art. Studies by the present inventors show no toxicity and no lethality for the present compounds at amounts up to 300 mg/kg in mice.

The compounds of the present invention can be administered as a single dosage per day, or as multiple dosages per day. When administered as multiple dosages, the dosages can be equal doses or doses of varying amount, based upon the time between the doses (i.e. when there will be a longer time between doses, such as overnight while sleeping, the dose administered will be higher to allow the compound to be present in the bloodstream of the patient for the longer period of time at effective levels). Preferably, the compound and compositions containing the compound are administered as a single dose or from 2-4 equal doses per day.

Suitable compositions containing the present compounds further comprise a physiologically acceptable carrier, such as water or conventional pharmaceutical solid carriers, and if desired, one or more buffers and other excipients.

### EXAMPLES

Having generally described this invention, a further understanding can be obtained by reference to certain specific examples which are provided herein for purposes of illustration only and are not intended to be limiting unless otherwise specified.

Chemistry

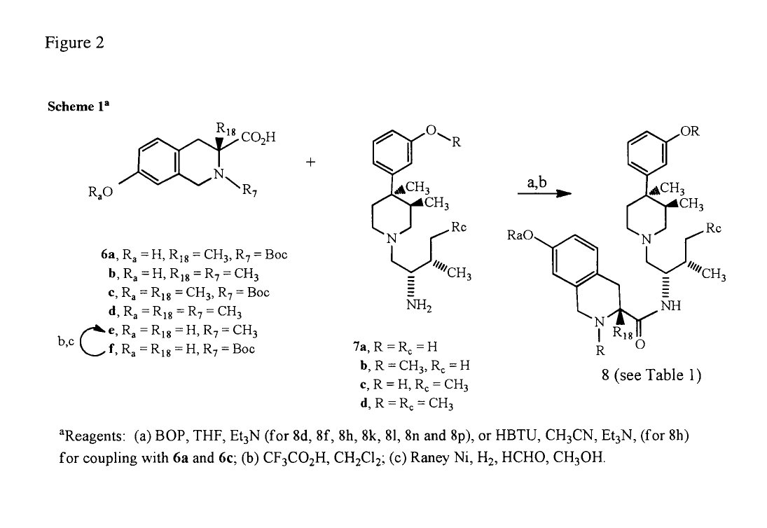

The structure of 3 was modified to introduce methyl groups at five different sites of the molecule (see Table 1 for exemplary structures): at the phenol moieties (Rₐ, R), on the linker of the phenylpiperidine to the tetrahydroisoquinoline carboxamide fragments (R_{c}), at the position alpha to the carboxamide moiety (R₁₈), and at the isoquinoline nitrogen (R₇). Analogues 8a-c were synthesized as previously reported.^{14,23} The synthesis of the new analogues 8d-p is shown in Scheme 1 (FIG. 2). Coupling of the appropriate 1,2,3,4-tetrahydroisoquinoline carboxylic acids 6a-e with 7a-d using benzotriazole-1-yloxy-tris(dimethylamino)phosphonium hexafluorophosphate (BOP) in tetrahydrofuran (THF) or O-(benzotriazol-1-yl)-N,N,N′,N′-tetramethyluronium hexafluorophosphate (HBTU) in acetonitrile (followed by removal of the Boc-protecting group with trifluoroacetic acid in methylene chloride when 6a and 6c were used) yielded 8d-p.

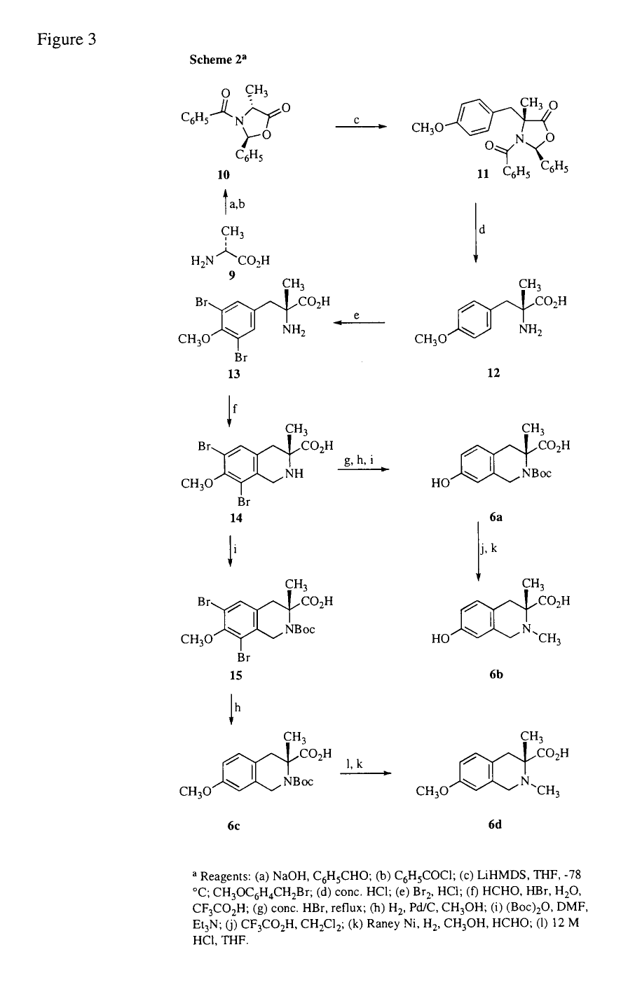

The tetrahydroisoquinoline carboxylic acids 6a-d needed for the synthesis of 8e, 8g, 8h, 8i, 8l, 8m, 8n, and 8o were prepared following the transformations outlined in Scheme 2 (FIG. 3). D-Alanine (9) was converted to the sodium salt using sodium hydroxide in ethanol, followed by conversion to the chiral oxazolidinone 10 by condensation with benzaldehyde under azeotropic distillation conditions and benzoylation using benzoyl chloride.²⁵ Alkylation of 10 with 4-methoxybenzyl bromide using lithium hexamethyldisilazide as the base at −78° C. proceeded with high diastereomeric selectivity to give the p-methoxybenzylated intermediate 11.²⁶ Acid hydrolysis of the chiral intermediate 11 gave the amino acid 12. Formation of the tetrahydroisoquinoline ring system was achieved via the Pictet-Spengler reaction. This was carried out by bromination of 12 to give 13 to protect the ortho positions of the methoxy group followed by treatment with hydrobromic acid and formaldehyde at 80° C. to give 14. Compound 14 was converted to 6a by treatment with concentrated hydrobromic acid to demethylate the 7-methoxy to a phenol, followed by catalytic debromination using palladium on carbon under hydrogen, and finally treatment with di-tert-butyl dicarbonate in dimethylformamide containing triethylamine to give 6a. The N-methyl analogue 6b was obtained by treating 6a with trifluoroacetic acid to give the free amine followed by reductive methylation using Raney nickel catalyst, hydrogen, and formaldehyde in methanol. Compounds 6c and 6d were obtained from 14 by protection as the tert-butoxycarbonyl ester using di-tert-butyl dicarbonate and then debromination using palladium on carbon as catalyst under hydrogen to give 6c. Removal of the Boc-protecting group from 6c using hydrochloric acid followed by reductive methylation using the same conditions as for 6b gave the N-methyl analogue 6d.

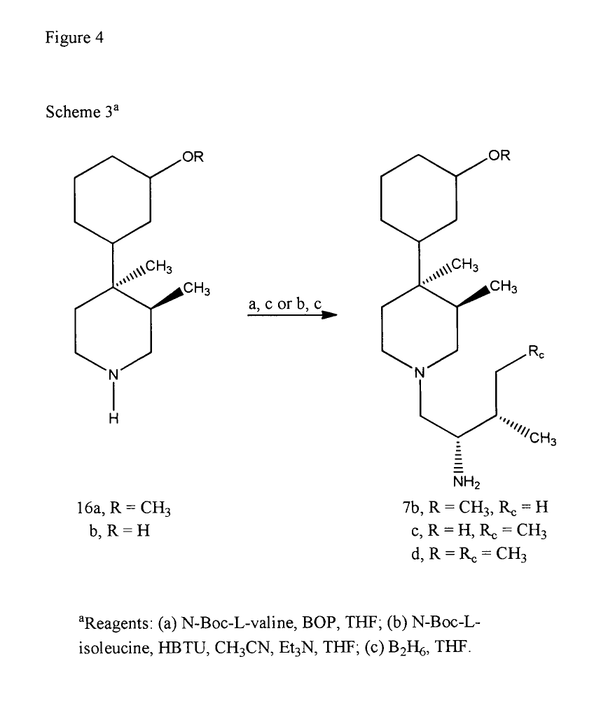

Compound 7b was synthesized by coupling N-Boc-L-valine with (3R,4R)-4-(3-methoxyphenyl)-3,4-dimethylpiperidine (16a)²⁷ using BOP in tetrahydrofuran followed by reduction with diborane in tetrahydrofuran (Scheme 3; FIG. 4). Coupling of 16b and 16a with N-Boc-L-isoleucine using HBTU in acetonitrile followed by reduction with diborane gave 7c and 7d, respectively. Compound 7a was synthesized as previously reported.²⁸

Pharmacology

Compounds 1, 3, and 8a-p were first evaluated at 10 μM for intrinsic activity in the [³⁵S]GTPγS binding assay at all three opioid receptors. As none of the compounds displayed measurable intrinsic activity at this concentration, they and the reference compound 1 were evaluated for functional antagonism and selectivity at the opioid receptors. These data were obtained by monitoring the ability of test compounds to inhibit stimulated [³⁵S]GTPγS binding produced by the selective agonists DAMGO (μ), DPDPE (δ), or U69,593 (κ) using cloned human opioid receptors expressed in CHO cells.²⁹ Agonist dose response curves were run in the presence or absence of a single concentration of test compound. Test compound assay concentrations ranged from 1-5000 nM, depending on their activity. The Kₑ values were calculated using the formula: Kₑ=[L]/DR−1, where [L] is the concentration of test compound and DR is the ratio of agonist EC₅₀ value in the presence or absence of test compound, respectively. At least two different concentrations of test compound were used to calculate the 1c, and the concentrations were chosen such that the agonist EC₅₀ exhibited at least a four-fold shift to the right and there was a clear upper asymptote to the agonist+compound concentration response curve. The Kₑ values along with those for the reference compound 1 are shown in Table 1.

The calculated log P, tPSA, and log BB values for compounds 1, 3, and 8a-p are given in Table 2. The log BB values were calculated using equation 6 (the Clark equation) given in reference 24. Topological polar surface areas (tPSA) and log P values were calculated using ChemAxon's Instant JChem® version 5.03 software.

Results and Discussion

Even though 3 (Kₑ=0.02 nm) was more potent as a κ-opioid receptor antagonist than any of the methylated analogues studied, many of the analogs were potent and selective κ antagonists. All of the monomethylated analogues 8a-8e, the dimethylated analogues 8f-8j, and the trimethylated analogues 8m retained subnanomolar potency at the κ-opioid receptor. All of the monomethylated compounds 8a-8e, the dimethylated compounds 8h and 8k, and the trimethylated compound 8n retained greater than 100-fold κ selectivity relative to the μ and δ receptors. The two most potent analogues were 8a (R₃═CH₃) and 8e (R₄═CH₃), both with Kₑ values of 0.03 nM at the ic-opioid receptor. Both compounds had 100-fold or greater selectivity for the κ receptor relative to the μ receptor. The κ selectivity for 8a and 8e relative to the δ receptor was 800 and 28,500, respectively. The N-methyl compound 8c (R₅═CH₃) with a Kₑ value of 0.16 nM at the κ-opioid receptor and 1313- and 3070-fold selectivity for the κ receptor relative to the μ and δ receptors was the most κ selective analogue of this new series. Compound 8b (R₂═CH₃) with a Kₑ value of 0.06 nM was 3 times less potent than 3, and with μ/κ and μ/δ ratios of 857 and 1970, it was also highly κ selective.

Compound 8d, with a methyl substituted at the 3-hydroxyl in the phenylpiperidine fragment, had only a two-fold decrease in potency for the ic receptor (Kₑ=0.037 nM) relative to 3.

Methylation of the alkyl side chain on the linker between the phenylpiperidine and tetrahydroisoquinoline carboxamide fragments (R₃) produced compounds 8a, 8f, 8i, 8j, and 8m that had increased potency at μ receptors compared to 3. This effect was most notable in the monomethyl substituted compound 8a, which had an eight-fold increase in potency at μ receptors compared to 3. These observations mirror those seen in previous studies where large substituents at this position increased μ receptor potency.²⁸

N-Methylation at the tetrahydroisoquinoline nitrogen to give the N-methyl 3 analogue 8c resulted in a reduction in potency at all receptor subtypes. At κ receptors, this modification consistently gave decreases in potency for all analogues 8j, 8k, 8o, and 8p. Nevertheless, analogues 8c and 8j with Kₑ values of 0.16 and 0.11 nM, respectively, were still highly potent κ antagonists.

In general it was observed that introduction of multiple methyl groups into the structure of 3 was detrimental for potency and selectivity at ic receptors. Compounds 81 and 8j with Kₑ values of 0.11 nM each at the κ receptor were the two most potent analogues with multiple methyl groups. The effect was more noticeable for compounds with three methyl substitutions. The most potent analogue containing three methyl groups was 8n, which had a 1c value of 0.52 nM at the κ receptor.

The calculated log P, tPSA, and log BB values for 1, 3, and 8a-p are given in Table 2.²⁴ In contrast to standard compound 1 (calculated log BB=−1.42), the calculated log BB for 3 and 8a-p (−0.07 to −0.55) are above the threshold proposed by Clark to indicate low blood brain barrier penetration. The calculated log BB values²⁴ show that all 16 methylated analogues would be expected to show enhanced brain penetration relative to 3. In the case of 8b for instance, monomethylation shifts the calculated log BB value positively by 0.22 log units (calculated log BBs for 3 and 8b are −0.55 and −0.33, respectively). This change in the relative concentration of drugs implies an approximately 66% increase in the concentration of the drug in the brain.

In summary, 16 analogues of 3 with methyl substituents at five different positions on the 3 structure were synthesized. Eleven of the analogues had sub-nanomolar κ values at the κ opioid receptor. The monomethylated analogues 8a, 8b, 8d, and 8e with Kₑ values of 0.03 to 0.06 nM were the most potent compounds. Even though the efficacy at the opioid receptor is not as good as that for 3, the calculated log BB values suggest that these analogues may have activity comparable to that of 3 in vivo.

### Experimental Section

¹H NMR spectra were determined on a Bruker 300 spectrometer using tetramethylsilane as an internal standard. Mass spectral data were obtained using a Finnegan LCQ electrospray mass spectrometer in positive ion mode at atmospheric pressure. Medium-pressure flash column chromatography was done on a CombiFlash Companion system using Teledyne Isco prepacked silica gel columns or using EM Science Silica Gel 60 Å (230-400 mesh). All reactions were followed by thin-layer chromatography using Whatman silica gel 60 TLC plates and were visualized by UV. Optical rotations were measured on an Auto Pol III polarimeter. All solvents were reagent grade. HCl in dry diethyl ether was purchased from Aldrich Chemical Co. and used while fresh before discoloration. CMA-80 is a mixture of 80% chloroform, 18% methanol, and 2% concentrated ammonium hydroxide. Purity of compounds (>95%) was established by elemental analysis. Elemental analyses were performed by Atlantic Microlab, Inc., Atlanta, Ga. Care should be used when using BOP in coupling reactions as it yields the carcinogenic byproduct HMPA.

### (2S,4R)-4-(4-Methoxybenzyl)-4-methyl-2-phenyl-3-(phenylcarbonyl)-1,3-oxazolidin-5-one (11)

Compound 10²⁵ (6.35 g, 0.023 mol) in 50 mL of THF at −78° C. was added over 20 min to a solution of LiHMDS in THF (25 mL of 1 M solution in THF). After 10 min, 1.1 eq. of 4-methoxybenzyl bromide (25 mmol, 5 mL) was added in one portion. The mixture was stirred at −78° C. for 3 h and then at room temperature overnight. Saturated NH₄Cl solution was added, the THF was removed in vacuo, Et₂O (100 mL) was added, and the phases were separated. The organic layer was washed with 50 mL of NaHCO₃ solution and brine. After drying (Na₂SO₄), filtration, and removal of the solvent, the residue was purified by chromatography using silica gel Isco column with 9% EtOAc in hexanes as eluent. Concentration of the product fractions gave 7.4 g (82%) of 11 as a white solid: mp 128-129° C. [α]²⁵_{D}=−260 (c 0.8, MeOH). ¹H NMR (CDCl₃) δ 7.27 (2H, d, J=8 Hz), 7.19-7.14 (2H, m), 7.09-7.05 (4H, m), 6.94 (d, 2H, J=8 Hz), 6.76-6.72 (m, 4H), 5.68 (s, 1H), 3.88 (d, 1H, J=12 Hz), 3.86 (s, 3H), 3.27 (d, 1H, J=12 Hz), 2.14 (s, 3H). ¹³C NMR 175.1, 169.4, 159.6, 136.7, 131.5, 130.1, 130.0, 128.8, 128.7, 128.2, 127.2, 126.3, 114.6, 90.7, 65.9, 55.8, 40.5, 24.6. ESIMS: m/z 402 (M+1, 100).

### O,α-Dimethyl-D-tyrosine (12)

Compound II (2.2 g, 0.0055 mol) was suspended in 20 mL of concentrated HCl solution. After nitrogen flush, the mixture was heated under reflux for 3 h. After filtration and removal of the HCl solution, the white precipitate was dried. ¹H NMR (CD₃OD) δ 7.24 (d, 2H, J=6 Hz), 6.91 (d, 2H, J=6 Hz), 3.77 (3H, s), 3.26 (d, 1H, J=14 Hz), 3.13 (d, 1H, J=14 Hz), 1.66 (s, 3H). ¹³C NMR 173.8, 161.3, 132.9, 115.9, 62.4, 56.4, 43.5, 23.2, MS (ESI) 210 (M+1). The product was used in the next step without purification.

### 3,5-Dibromo-O,α-dimethyl-D-tyrosine (13)

To a solution of compound 12 from above in distilled water (20 mL), 12 M HCl (4 mL) was added. The reaction mixture was cooled to 5° C., and bromine (2.1 mL, 41 mmol) was injected into the stirred solution. After 15 min, Ni gas was passed through the reaction mixture until the product precipitated. APCIMS: m/z 366 (M+1, 100). The product was used in the next step without purification.

### (3R)-6,8-Dibromo-7-methoxy-3-methyl-1,2,3,4-tetrahydroisoquinoline-3-carboxylic Acid (14)

Compound 13 from above (assumed to be 4.8 mmol) was added to trifluoroacetic acid (5 mL). HBr (33% in acetic acid, 0.9 mL, 4.8 mmol) was added dropwise to the reaction mixture under a nitrogen atmosphere. After the addition of the acid, formaldehyde (8.64 mmol, 260 mg, 0.7 mL) was added dropwise and the mixture stirred at 70-80° C. for 17 h. The reaction mixture was cooled, dried, and concentrated. APCIMS: m/z 378 (M+1). The product was used in the next step without purification.

### (3R)-6,8-Dibromo-2-(tert-butoxycarbonyl)-7-methoxy-3-methyl-1,2,3,4-tetrahydroisoquinoline-3-carboxylic Acid (15)

The crude compound 14 reported above (assumed to be 4.8 mmol) was dissolved in DMF (7 mL) and water (2 mL). Triethylamine (1.01 g, 0.01 mol) was added, followed by di-tert-butyl dicarbonate (1.57 g, 0.007 mol). The reaction mixture was stirred at room temperature for 4 h and then concentrated to dryness. The resulting residue was treated with water (30 mL) and EtOAc (30 mL). KHSO₄ (2 g) was added to the mixture (pH=2), and the organic layer was separated, dried, and concentrated. The product was purified by chromatography on silica gel (Isco column), using 35% EtOAc in hexanes as eluent to afford 500 mg of 15 (22% from 13) as a syrup. ¹H NMR (CD₃OD) δ 7.55 (s, 1H), 4.84 (d, 1H, J=16 Hz), 4.54 (d, 1H, J=16 Hz), 3.85 (s, 3H), 3.19 (d, 1H, J=16 Hz), 2.92 (d, 1H, J=16 Hz), 1.47 (s, 9H), 1.42 (s, 3H). ¹C NMR 177.7, 154.7, 138.1, 135.4, 132.9, 118.6, 117.9, 62.5, 62.0, 46.1, 41.7, 29.1, 28.3, 23.9. ESIMS: m/z 478 (M+1).

### (3R)-2-(tert-Butoxycarbonyl)-7-hydroxy-3-methyl-1,2,3,4-tetrahydroisoquinoline-3-carboxylic Acid (6a)

A suspension of 14 (5.00 g, 0.012 mol) in 75 mL of 48% aqueous HBr was heated to reflux for 5 h. The solution was then evaporated to dryness under reduced pressure and dissolved in 30 mL of MeOH and 7.00 mL of Et₃N (0.05 mol). This solution was added to 300 mg of 10% Pd on carbon and shaken for 12 h in a Parr hydrogenator under 60 psig Hi. The suspension was filtered and the solvents removed under reduced pressure to leave a solid product (containing the product and triethylammonium salts) with a mass of 9.77 g. This solid was dissolved in 15 mL of H₂O, 40 mL of DMF, and 4.78 mL (34.29 mmol) of Et₃N. Into this solution, di-tert-butyl dicarbonate (2.1 mL, 22.26 mmol) was introduced and the mixture stirred for 10 h. The solution was reduced to 1/10 of its volume under reduced pressure and partitioned between 30 mL of H₂O and 30 mL of EtOAc. The water layer was extracted with EtOAc (3×15 mL). The pooled organic extracts were washed once each with 10 mL of H₂O, 10 mL of brine, dried over MgSO₄, filtered, and concentrated to dryness to yield 3.42 g of 6a as a foam that was pure by NMR. ¹H NMR (CDCl₃) δ 7.17 (d, 1H, J=8.1 Hz), 6.73 (m, 2H), 4.60-4.41 (2d, 2H), 3.12 (d, 1H, J=14.7 Hz), 2.78 (d, 1H, J=14.7 Hz), 1.56-1.24 (2s, 12H). ESIMS: m/z 207 (M+1-Boc).

### (R)-7-Hydroxy-2,3-dimethyl-1,2,3,4-tetrahydroisoquinoline-3-carboxylic Acid (6b) Triethylammonium Salt

The Boc-protected isoquinoline 6a (534 mg, 1.74 mmol) was dissolved in 5 mL of a 1:1 mixture of CF₃CO₂H/CH₂Cl₂ and stirred overnight. The solvents were removed under reduced pressure and the residue suspended in 2 mL of water. The pH of the solution was adjusted to 7 by addition of saturated NaHCO₃. To this solution was added 300 mg of Raney Ni slurry in MeOH using a spatula along with 1 mL of a 37% solution of formaldehyde in water (13.4 mmol), and the resulting suspension was stirred under 1 atm of H₂ overnight. The suspension was filtered, and the solvents were removed under reduced pressure to yield a residue that was subjected to silica gel flash-column chromatography. Elution with CHCl₃/MeOH/NH₄OH (60:30:10) afforded 384 mg of the ammonium salt of 6b after removal of solvents. The triethylammonium salt of 6b was prepared by addition of 5 mL of Et₃N to a solution of the compound in 2 mL of MeOH, followed by removal of the volatiles: mp>220° C. ¹H NMR (CD₃OD) δ 7.07 (d, 1H, J=8 Hz), 7.77 (d, 1H), 6.58 (s, 1H), 4.43 (bd, 1H), 4.31 (bd, 1H), 3.37 (d, 1H), 3.20 (m, 9H), 2.95 (d, 1H, J=14.7 Hz), 1.52 (s, 3H), 1.25 (t, 9H), 2.88 (m, 1H), 2.75 (m, 1H). ESIMS: m/z 222 (M+1, 100).

### (3R)-2-(tert-Butoxycarbonyl)-7-methoxy-3-methyl-1,2,3,4-tetrahydroisoquinoline-3-carboxylic Acid (6c)

Triethylamine (3 mmol, 0.42 mL) and 10% Pd/C (20 mg) were added to 15 (337 mg, 1.05 mmol) in MeOH (5 mL). This mixture was shaken for 90 min under 40 psig of H₂ in a Parr apparatus. The mixture was then filtered and concentrated under reduced pressure to give 6c in quantitative yield. An analytical sample was prepared by recrystallization from EtOAc-hexanes: mp 191° C. dec. ¹H NMR (CD₃OD) δ 7.10 (d, 1H, J=8 Hz), 6.82 (m, 3H), 4.69 (d, 1H), 4.40 (d, 1H), 3.18 (d, 1H, J=14.7 Hz), 2.79 (d, 1H, J=14.7 Hz), 1.46 (s, 9H), 1.39 (s, 3H). ESIMS: m/z 322 (M+1, 100).

### (3R)-7-Methoxy-2,3-dimethyl-1,2,3,4-tetrahydroisoquinoline-3-carboxylic Acid (6d) Triethylammonium Salt

At 0° C., 6c (266 mg, 1.13 mmol) was dissolved in 5 mL of THF and 2 mL of 12 M HCl. After this solution was stirred for 4 h, the solvents were removed under reduced pressure. The residue was dissolved in 5 mL of MeOH. Into this solution were added 0.12 mL of Et₃N, 0.5 mL of 37% formaldehyde in H₂O, and 0.3 mL of Raney Ni slurry in MeOH. The mixture was stirred overnight under an atmosphere of Hi, filtered, and the solvents removed under reduced pressure to yield a residue that contained the title compound and Et₃N.HCl. This residue was used without further purification. ¹H NMR (CD₃OD) δ 7.13 (d, 1H), 6.88 (d, 1H), 6.75 (s, 1H), 4.57 (d, 1H), 4.32 (d, 1H) 3.77 (s, 3H), 3.41 (d, 1H, J=14.7 Hz), 3.21 (q, 6H), 2.99 (d, 1H), 2.90 (s, 3H), 1.53 (s, 3H), 1.31 (t, 9H). ESIMS: m/z 322 (M+1, 100).

### (3R)-7-Hydroxy-2-methyl-1,2,3,4-tetrahydroisoquinoline-3-carboxylic Acid (6e) Hydrochloride

Compound 6r³⁰ (1.087 g, 0.0034 mol) was suspended in 15 mL of CH₂Cl₂, and the mixture was cooled to 0° C. Into this solution was added 7 mL of CF₃COOH, and the mixture was stirred for 6 h. The solvents were removed under reduced pressure, and the residue was suspended in 100 mL of MeOH and 5 mL of formalin. Into this suspension was added 1 g of a slurry of Raney Ni in MeOH using a spatula. The mixture was stirred under an atmosphere of H₂ for 5 h and was filtered through Celite. To the filtered solution was added 10 mL of a 2 M solution of HCl in ethanol. The solvents were removed under reduced pressure, and the residue was recrystallized from MeOH to give 879 mg (64%) of 6e.HCl as a white powder: mp>220° C. ¹H NMR (d₆-DMSO) δ 9.59 (s, 1H), 7.09 (d, 1H, J=8.4 Hz), 6.73 (m, 1H), 6.59 (s, 1H), 4.53 (b, 1H), 4.38 (b, 2H), 3.31 (dd, 1H, J=5.7 Hz), 3.16-3.07 (m, 1H), 2.91 (s, 3H). ESIMS: m/z 208 (M+1, 100).

### (2S)-1-[(3R,4R)-4-(3-Methoxyphenyl)-3,4-dimethylpiperidin-1-yl]-3-methylbutan-2-amine (7b)

To a heterogeneous solution of (3R,4R)-4-(3-methoxyphenyl)-3,4-dimethylpiperidine²⁷ (41.1 g, 0.161 mol), N-Boc-L-valine (34.9 g, 0.161 mol), BOP reagent (71.0 g, 0.161 mol) in THF (450 mL) was added triethylamine (51.9 g, 0.513 mol) in THF (50 mL). The reaction mixture became homogeneous within 5 min after addition of Et₃N. The reaction was stirred for 4 h at room temperature than added to ether (500 mL)/H₂O (300 mL). The organic layer was separated, washed with saturated NaHCO₃ then brine, and separated. The extracts were dried (Na₂SO₄) and concentrated in vacuo to afford an off-white solid. This material was purified by silica gel column chromatography, eluting with 70% hexanes in EtOAc to yield 63.6 g (94%) of a white amorphous solid.

Diborane (260 mL, 1.0 M in THF, 0.260 mol) was added to the material described above (54.6 g, 0.130 mol) in THF (350 mL). The reaction mixture was stirred under N2 at reflux for 2 h. The slightly heterogeneous reaction mixture was cooled to room temperature, and 6 N HCl was added (initially cautiously). After stirring at reflux for 2 h, the mixture was concentrated in vacuo and diluted with water. The reaction mixture was made basic with solid Na₂CO₃ and extracted with CH₂Cl₂. The organic layer was separated, dried (Na₂SO₄), and concentrated in vacuo to afford 44 g (100%) of a thick oil. ¹H NMR (CDCl₃) δ 7.21 (t, 1H), 6.88 (d, 1H, J=8.1 Hz), 6.83 (s, 1H), 6.71 (d, 1H), 3.81 (s, 3H), 2.77 (m, 1H), 2.63-2.13 (m, 8H), 2.10 (bm, 1H), 2.00 (m, 1H), 1.60 (m, 1H), 1.41 (s, 3H), 0.91 (m, 7H), 0.76 (d, 3H, J=7.2 Hz). ESIMS: m/z 305 (M+H⁺, 100). Anal. (C₁₉H₃₂N₂O) C, H, N.

### 3-{(3R,4R)-1-[(2S,3S)-2-Amino-3-methylpentyl]-3,4-dimethylpiperidin-4-yl}phenol (7c).²³

3-[(3R,4R)-3,4-Dimethylpiperidin-4-yl]phenol (2.42 g, 11.79 mmol) and L-Boc-Ile (2.73 g, 11.79 mmol) were stirred in 30 mL of CH₃CN and the solution cooled to 0° C. Into this solution, HBTU (4.47 g, 11.79 mmol) was added followed by Et₃N (3.3 mL, 23.57 mmol). The solution was stirred for 2 h and was then partitioned between 60 mL of EtOAc and 20 mL of H₂O. The organic layer was washed with saturated NaHCO₃ (10 mL×3) and brine (10 mL). The solvent was dried over Na₂SO₄, filtered, and removed under reduced pressure. Flash column chromatography on silica gel eluting with a solvent gradient (80% hexanes in EtOAc to 66% hexanes in EtOAc) gave fractions that contained 3.39 g of pure amide. The amide (3.37 g, 8.33 mmol) was dissolved in 20 mL of dry THF, and 16.67 mL of a 1 M solution of BH₃ in TI-IF was added. The solution was heated at reflux for 3 h, cooled to ambient temperature, and carefully added to 3 mL of H₂O. Then 7 mL of conc. HCl was added. The mixture was heated at reflux for 2 h, and the volume of the reaction was reduced to one-third under reduced pressure. The remaining mixture was made basic by addition of solid NaHCO₃ and extracted thoroughly with a 4:1 mixture of CH₂Cl₂/THF. The pooled extracts were washed once with 20 mL of H₂O, dried over MgSO₄, filtered, and concentrated to give 2.50 g (70%) of a clear oil that slowly crystallized. An analytical sample was prepared by recrystallization from EtOAc: mp 150-153° C. ¹H NMR (CDCl₃) δ 7.13 (t, 1H), 6.80 (m, 1H), 6.71 (s, 1H), 6.64 (d, 1H), 2.82-2.78 (m, 3H), 2.74-2.52 (m, 4H), 2.42-2.26 (m, 6H), 1.97 (m, 1H), 1.54 (m, 2H), 1.49-1.38 (m, 1H), 1.31 (s, 3H), 1.27-1.19 (m, 1H), 0.89 (t, 6H), 0.77 (d, 3H, J=6.9 Hz).). ESIMS: m/z 305 (M+H⁺, 100). Anal. (C₁₉H₃₂N₂O) C, H, N.

### (2S,3S)-1-[(3R,4R)-4-(3-Methoxyphenyl)-3,4-dimethylpiperidin-1-yl]-3-methylpentan-2-amine (7d)

(3R,4R)-4-(3-Methoxyphenyl)-3,4-dimethylpiperidine²⁷ (533 mg, 2.43 mmol) and Boc-L-Ile (562 mg, 2.43 mmol) were stirred in 20 mL of CH₃CN, and the solution was cooled to 0° C. Into this solution was added HBTU (922 mg, 2.43 mmol) followed by Et₃N (0.7 mL, 4.87 mmol). The solution was stirred for 2 h and was then partitioned between 30 mL of EtOAc and 10 mL of H₂O. The organic layer was washed with saturated NaHCO₃ (7 mL×3) and brine (5 mL) solutions. The solvent was dried over Na₂SO₄, filtered, and removed under reduced pressure. Flash column chromatography on silica gel eluting with 83% hexanes in EtOAc gave fractions that after removal of solvent yielded 680 mg of pure amide. The amide (675 mg, 1.56 mmol) was dissolved in 20 mL of dry THF and 3.12 mL of a 1 M solution of BH₃ in THF was added. The solution was heated at reflux for 3 h, cooled to room temperature, and then 1 mL of H₂O was added carefully followed by 3 mL of conc. HCl. The mixture was heated at reflux for 2 h, and the volume of the reaction was reduced to one-third under reduced pressure. The remaining mixture was made basic by addition of NaHCO₃ and extracted thoroughly with CH₂Cl₂. The pooled extracts were washed once with 10 mL of H₂O, dried over MgSO₄, filtered, and the solvents removed to give 540 mg (70%) of a clear oil. ¹H NMR (CD₃OD) δ 7.20 (t, 1H, ArH), 6.89 (m, 1H, ArH), 6.82 (s, 1H, ArH), 6.72 (m, 1H, ArH), 3.77 (s, 3H, CH₃OAr), 2.88-2.25 (m, 9H), 2.03 (m, 1H), 2.57-2.40 (m, 3H), 1.30 (d, 3H, CH₃, J=6.6 Hz).), 1.28-1.10 (m, 1H), 0.96-0.89 (m, 7H), 1.60-1.70 (dd, 3H, CH₃). EIMS: m/z 319 (M+H⁺, 100). Anal. (C₂₀H₃₄N₂O) C, H, N.

General procedures for the preparation of compounds 8d-p.

(a) BOP Coupling Procedure:

A phenylpiperidine 7 (1 eq.) was dissolved along with a tetrahydroisoquinoline 6 (1.05 eq.) in 10 mL of dry THF and cooled to 0° C. Into this flask was introduced BOP (1.05 eq.) dissolved in 5 mL of dry THF. Immediately afterwards Et₃N (1.05 eq.) was added, and the solution was warmed to room temperature and allowed to stir for 3 h. The solution was added to 30 mL of saturated NaHCO₃. The resulting mixture was extracted 3× with 10 mL of EtOAc. The pooled organic solvents were washed once with 5 mL of water and dried over MgSO₄. The mixture was then separated by flash chromatography on silica gel. For the reactions employing Boc-protected tetrahydroisoquinolines, the crude coupling mixture was dissolved in 10 mL of a 20% CF₃CO₂H solution in CH₂Cl₂ and stirred overnight. The solvents were removed and the crude product stirred in 10 mL of saturated NaHCO₃ and 10 mL of EtOAc. The layers were separated, and the aqueous layer was extracted 2× with 5 mL of EtOAc. The pooled EtOAc extracts were washed once with 3 mL of brine, dried over Na₂SO₄, filtered, and concentrated under reduced pressure to yield a crude residue. When needed, the impure compound was purified by preparative thick layer chromatography. The dihydrochloride salts were formed by dissolving the freebase in 5 mL of EtOH followed by addition of 5 mL of 2 M HCl in EtOH and evaporation of the solution under reduced pressure.

(b) HBTU Coupling Procedure:

A phenylpiperidine 7 (1 eq.) was dissolved along with a tetrahydroisoquinoline 6 (1.05 eq.) in 15 mL of a 50% solution of THF in CH₃CN and cooled to 0° C. Into this flask was introduced HBTU (1.05 eq.) dissolved in 10 mL of CH₃CN. Immediately afterwards Et₃N (1.05 eq.) was added, and the solution was warmed to room temperature and allowed to stir for 3 h. To the reaction solution was added 30 mL of saturated NaHCO₃. The resulting mixture was extracted three times with 10 mL of EtOAc. The pooled organic solvents were washed once with 5 mL of water and dried over MgSO₄. The mixture was then separated by chromatography. For the reactions employing Boc-protected tetrahydroisoquinolines, the crude coupling mixture was dissolved in 10 mL of a 20% CF₃CO₂H solution in CH₂Cl₂ and stirred overnight. The solvents were removed and the crude product stirred in 10 mL of saturated NaHCO₃ and 10 mL of EtOAc. The layers were separated, and the aqueous layer was extracted 2× with 5 mL EtOAc. The pooled EtOAc extracts were washed once with 3 mL of brine, dried over Na₂SO₄, filtered, and concentrated under reduced pressure to yield a crude residue. When needed, the impure compound was purified by preparative thick layer chromatography. The dihydrochloride salts were formed by dissolving the freebase in 5 mL of EtOH followed by addition of 5 mL of 2 M HCl in EtOH and evaporation of the solvents under reduced pressure.

### (3R)-7-Hydroxy-N-[(1S)-1-{[(3R,4R)-4-(3-methoxyphenyl)-3,4-dimethylpiperidin-1-yl]methyl}-2-methylpropyl]-1,2,3,4-tetrahydroisoquinoline-3-carboxamide (8d) Dihydrochloride

General procedure (a) was employed using 100 mg (0.328 mmol) of 7b and 112 mg (0.382 mmol) of 6f to afford 65 mg (35%) of the freebase. ¹H NMR (CD₃OD) δ 7.26 (t, 1H, J=8.1 Hz), 7.00 (d, 1H, J=8.1 Hz), 6.91 (d, 1H, J=8.1 Hz), 6.86-6.75 (m, 2H), 6.63 (s, 1H), 4.30-4.37 (m, 3H), 3.50 (m, 2H), 3.15-3.11 (m, 1H), 2.80-2.71 (m, 1H), 2.45 (m, 1H), 1.93-1.87 (m, 1H), 1.49 (s, 3H), 1.29-1.13 (m, 1H), 1.07 (2d, 6H), 0.85 (d, 3H). The hydrochloride salt synthesized by the general procedure had mp>220° C. dec. [α]_{D}²⁵+69° (c 0.35, MeOH). ¹H NMR (CD₃OD) δ 7.31 (t, 1H, J=9 Hz), 7.12 (d, 1H, J=9 Hz), 6.95 (d, 1H), 6.86-6.73 (m, 3H), 6.63 (s, 1H), 4.40 (d, 1H), 4.34 (d, 1H), 4.27 (m, 2H), 3.81 (s, 3H), 3.63 (d, 1H), 3.60-3.24 (m, 6H), 3.20 (d, 1H), 2.63 (dt, 1H), 2.43 (m, 1H), 1.95 (m, 1H), 1.48 (s, 3H), 1.03-0.87 (m, 3H), 0.83 (d, 3H, J=9 Hz), 0.80-0.68 (m, 6H). ESIMS: m/z 480 (M+1, 50). Anal. (C₂₉H₄₃Cl₂N₃O₃.2H₂O) C, H, N.

### (3R)-7-Hydroxy-N-[(1S)-1-[(3R,4R)-4-(3-hydroxyphenyl)-3,4-dimethylpiperidin-1-yl]methyl}-(2-methylpropyl]-3-methyl-1,2,3,4-tetrahydroisoquinoline-3-carboxamide (8e) Dihydrochloride

General procedure (b) was employed using 100 mg (0.344 mmol) of 7a and 111 mg (0.361 mmol) of 6a to afford 65 mg (35%) of the freebase after separation by preparative TLC eluting with 1:1 CMA-80/CH₂Cl₂. ¹H NMR (CD₃OD) δ 7.09 (t, 1H, J=8.4 Hz), 6.87 (d, 1H, J=8.4 Hz), 6.70 (m, 2H), 6.55 (m, 2H), 6.47 (s, 1H), 4.02 (d, 1H), 3.77 (m, 2H), 3.16 (d, 1H), 2.74-2.33 (m, 7H), 2.14 (dt, 1H), 1.89-1.75 (m, 2H), 1.47 (d, 1H), 1.38 (d, 3H), 1.26-1.17 (m, 7H), 0.82 (t, 6H), 0.58 (d, 3H). The hydrochloride salt synthesized by the general procedure had mp>220° C. dec. [α]_{D}²⁵+47.2° (c 1, MeOH). ¹H NMR (CD₃OD) δ 7.18 (m, 2H), 6.75 (m, 3H), 6.62 (m, 1H), 4.42 (d, 1H), 4.27 (d, 1H), 4.25 (m, 1H), 3.67-3.30 (m, 6H), 3.18 (d, 1H), 2.63 (dt, 1H), 2.39 (m, 1H), 1.90 (d, 1H), 1.85-1.60 (m, 1H), 1.79 (s, 3H), 0.85 (d, 3H, J=9 Hz), 0.68 (2d, 6H). ESIMS: m/z 480 (M+1, 50). Anal. (C₂₉H₄₁Cl₂N₃O₃.2H₂O) C, H, N.

### (3R)-7-Hydroxy-N-[(1S,2S)-1-{[(3R,4R)-4-(3-methoxyphenyl)-3,4-dimethylpiperidin-1-yl]methyl}-2-methylbutanyl]-1,2,3,4-tetrahydroisoquinoline-3-carboxamide (8f) Dihydrochloride

General procedure (a) was employed using 120 mg (0.377 mmol) of 7d and 116 mg (0.396 mmol) of 6f to afford 50 mg (27%) of the freebase after separation by preparative TLC eluting with 1:1 CMA-80/Et₂O, NMR (CD₃OD) δ 7.19 (t, 1H), 6.87-6.81 (m, 2H), 6.80 (s, 1H), 6.69 (ds, 1H), 6.58 (ds, 1H), 6.47 (s, 1H), 4.06 (m, 1H), 3.77 (dd, 2H), 3.75 (s, 3H), 3.50 (dd, 1H), 2.82 (dd, 1H), 2.78-2.70 (m, 2H), 2.65-2.39 (m, 5H), 2.27 (dt, 1H), 1.99 (m, 1H), 1.70-1.50 (m, 4H), 1.40 (m, 5H), 1.22-1.03 (m, 2H), 0.8 (m, 9H), 0.69 (d, 3H). The hydrochloride salt synthesized by the general procedure had mp>220° C. dec. [α]_{D}²⁵+95.9° (c 0.71, MeOH). ¹H NMR (CD₃OD) δ 7.30 (t, 1H, J=9 Hz), 7.12 (d, 1H, J=9 Hz), 6.91 (d, 1H, J=9 Hz), 6.89-6.73 (m, 2H), 6.62 (s, 1H), 4.40-4.20 (m, 3H), 3.89 (d, 1H), 3.81 (s, 3H), 3.67-3.23 (m, 7H), 3.12 (m, 1H), 2.82 (dt, 1H), 2.45 (m, 1H), 1.92 (d, 1H), 1.70 (m, 1H), 1.55 (m, 1H), 1.50 (s, 3H), 1.42 (m, 1H), 1.31 (m, 1H), 1.20 (m, 1H), 1.10-0.89 (m, 9H). ESIMS: m/z 494 (M+1, 80). Anal. (C₃₀H₄₅Cl₂N₃O₃H₂O) C, H, N.

### (3R)-7-Methoxy-N-[(1S)-1-[(3R,4R)-4-(3-hydroxyphenyl)-3,4-dimethylpiperidin-1-yl]methyl}-(2-methylpropyl]-3-methyl-1,2,3,4-tetrahydroisoquinoline-3-carboxamide (8 g) Dihydrochloride

General procedure (b) was employed using 172 mg (0.593 mmol) of 7a and 200 mg (0.622 mmol) of 6c to afford 250 mg of the freebase after isolation (68% yield): mp 210-212° C. ¹H NMR (CD₃OD) δ 7.09 (t, 1H, J=8.1 Hz), 6.73-6.57 (m, 2H), 4.13 (d, 1H), 3.95-3.87 (m, 2H), 3.64 (s, 3H), 3.25 (d, 2H), 2.68 (m, 1H), 2.65-2.50 (m, 2H), 2.40 (m, 4H), 2.18 (dt, 1H), 1.82 (m, 2H), 1.52 (d, 1H), 1.37 (s, 3H), 1.24 (s, 3H), 0.85 (2d), 0.47 (d, 3H). The hydrochloride salt synthesized by the general procedure had mp 210-212° C. dec. [α]_{D}²⁵+15° (c 1.2, MeOH). ¹H NMR (CD₃OD) δ 7.17-6.74 (m, 5H), 6.61 (m, 1H), 4.44 (d, 1H), 4.25 (d, 1H), 4.23 (m, 1H), 3.79 (s, 3H), 3.65-3.29 (m, 6H), 3.17 (d, 1H), 2.63 (dt, 1H), 2.40 (m, 1H), 1.91 (d, 1H), 1.84-1.59 (m, 1H), 1.79 (s, 3H), 0.84 (d, 3H, J=9 Hz), 0.67 (2d, 6H). ESIMS: m/z 494 (M+1, 100). Anal. (C₃₀H₄₅Cl₂N₃O₃H₂O) C, H, N.

### (3R)-7-Hydroxy-N-[(1S)-1-{[(3R,4R)-4-(3-methoxyphenyl)-3,4-dimethylpiperidin-1-yl]methyl}-2-methylpropyl]-3-methyl-1,2,3,4-tetrahydroisoquinoline-3-carboxamide (8h) Dihydrochloride

General procedure (b) was employed using 100 mg (0.328 mmol) of 7b and 120 mg (0.390 mmol) of 6a to afford 27 mg (17%) of the freebase after separation by preparative TLC eluting with 75:1 EtOAc/Et₃N. ¹H NMR (CD₃OD) δ 7.17 (t, 1H), 6.79-6.90 (m, 2H), 6.70 (m, 1H), 6.56 (m, 1H), 6.48 (s, 1H), 4.00 (d, 1H), 3.87 (m, 2H), 3.78 (s, 3H), 3.17 (d, 1H), 2.72-2.28 (m, 7H), 2.15 (dt, 1H), 1.89 (m, 1H), 1.79 (sextet, 1H), 1.50 (d, 1H), 1.38-1.20 (m, 7H), 1.10-0.77 (m, 7H), 0.56 (d, 3H). The hydrochloride salt synthesized by the general procedure had mp>220° C. dec. [α]_{D}²⁵+49.8° (c 0.45, MeOH). ¹H NMR (CD₃OD) 7.30 (t, 1H, J=9 Hz), 7.13 (d, 1H, J=9 Hz), 6.94 (d, 1H, J=9 Hz), 6.85-6.74 (m, 3H), 6.63 (s, 1H), 4.41 (d, 1H), 4.35 (d, 1H), 4.27 (m, 1H), 3.81 (s, 3H), 3.63 (d, 1H), 3.60-3.25 (m, 6H), 3.20 (d, 1H), 2.63 (dt, 1H), 2.45 (m, 1H), 1.95 (m, 1H), 1.78 (s, 3H), 1.48 (s, 3H), 1.05-0.89 (m, 3H), 0.83 (d, 3H, J=9 Hz), 0.80-0.68 (m, 6H). ESIMS: m/z 494 (M+1, 80). Anal. (C₃₀H₄₅Cl₂N₃O₃H₂O) C, H, N.

### (3R)-7-Hydroxy-N-[(1S,2S)-1-{[(3R,4R)-4-(3-hydroxyphenyl)-3,4-dimethylpiperidin-1-yl]methyl}-2-methylbutanyl]-3-methyl-1,2,3,4-tetrahydroisoquinoline-3-carboxamide (81) Dihydrochloride

General procedure (a) was employed using 104 mg of 7d (0.341 mmol) and 110 mg (0.358 mmol) of 6a to afford 29 mg of the freebase after separation by preparative TLC eluting with 1:1 CMA-80/CH₂Cl₂ (17% yield). ¹H NMR (CD₃OD) δ 7.19 (t, 1H), 6.79 (d, 1H, J=8.1 Hz), 6.77-6.66 (m, 2H), 6.58 (m, 2H), 6.48 (s, 1H), 4.09 (q, 1H), 4.02-3.95 (d, 1H), 3.93-3.8 (m, 2H), 3.15 (d, 2H), 2.70-2.50 (m, 3H), 2.49-2.32 (m, 3H), 2.15 (dt, 1H), 1.88 (m, 1H), 1.62-1.3 (m, 11H), 0.91-0.79 (m, 9H), 0.58 (d, 3H, CH₃). The hydrochloride salt synthesized by the general procedure had mp>220° C. dec. [α]_{D}²⁵ +42.2° (c 0.51, MeOH). ¹H NMR (CD₃OD) δ 7.20 (t, 1H, J=9 Hz), 7.13 (d, 1H, J=9 Hz), 6.80-6.75 (m, 2H), 6.69-6.64 (m, 2H), 4.41 (d, 1H), 4.30 (d, 1H), 4.28 (m, 1H), 3.64-3.32 (m, 7H), 3.17 (d, 1H), 2.63 (dt, 1H), 2.40 (m, 1H), 1.92 (d, 1H), 1.76 (s, 3H), 1.47 (m, 4H), 1.20 (m, 2H), 0.92-0.71 (m, 9H). ESIMS: m/z 494 (M+1, 80). Anal. (C₃₀H₄₅Cl₂N₃O₃H₂O) C, H, N.

### (3R)-7-Hydroxy-N-[(1S,2S)-1-{[(3R,4R)-4-(3-hydroxyphenyl)-3,4-dimethylpiperidin-1-yl]methyl}-2-methylbutanyl]-2-methyl-1,2,3,4-tetrahydroisoquinoline-3-carboxamide (8j) Dihydrochloride

General procedure (a) was employed using 130 mg (0.427 mmol) of 7c and 109 mg (0.448 mmol) of 6e to afford 55 mg (25%) of the freebase after separation by preparative TLC eluting with 2:1 CMA-80/CH₂Cl₂. ¹H NMR (CD₃OD) δ 7.19 (t, 1H, J=8.1 Hz), 6.90 (d, 1H, J=8.1 Hz), 6.73 (m, 2H), 6.59 (m, 2H), 6.51 (s, 1H), 4.09 (q, 1H), 3.98 (m, 1H), 3.84 (d, 1H), 3.50 (d, 1H), 3.13 (t, 1H), 2.99 (m, 1H), 2.88 (m, 1H), 2.75 (m, 1H), 2.55 (m, 2H), 2.45 (s, 3H), 2.37 (m, 2H), 2.23 (m, 1H) 1.94 (m, 2H), 1.63 (m, 1H), 1.50 (m, 2H), 1.62-1.3 (m, 11H), 0.80-1.0 (m, 9H), 0.7 (d, 3H, CH₃). The hydrochloride salt synthesized by the general procedure had mp 180° C. dec. [α]_{D}²⁵+84.3° (c 0.6, MeOH). ¹H NMR (CD₃OD) δ 7.19-7.11 (m, 2H), 6.89-6.65 (m, 4H), 4.50 (m, 2H), 4.32 (m, 1H), 3.73 (d, 1H), 3.55-3.16 (m, 8H), 3.08 (s, 3H), 2.78 (dt, 1H), 2.39 (m, 1H), 1.86 (d, 1H, J=15 Hz), 1.68 (m, 1H), 1.51-1.40 (m, 4H), 1.21 (m, 2H), 1.02 (d, 3H, J=6 Hz), 0.97-0.80 (m, 6H). ESIMS: m/z 494 (M+1, 100). Anal. (C₃₀H₄₅O₂N₃O₃H₂O) C, H, N.

### (3R)-7-Hydroxy-N-[(1S)-1-{[(3R,4R)-4-(3-methoxyphenyl)-3,4-dimethylpiperidin-1-yl]methyl}-2-methylpropyl]-2-methyl-1,2,3,4-tetrahydroisoquinoline-3-carboxamide (8k) Dihydrochloride

General procedure (a) was employed using 120 mg (0.394 mmol) of 7b 86 mg (0.414 mmol) of 6e to afford 67 mg (35%) of the freebase after separation by preparative TLC eluting with 2:1:1 CMA-80/EtOAc/hexanes. ¹H NMR (CD₃OD) δ 7.20 (t, 1H, J=8.1 Hz), 6.91 (d, 1H, J=8.1 Hz), 6.86 (d, 1H, J=8.1 Hz), 6.81 (s, 1H), 6.71 (dd, 1H), 6.59 (dd, 1H), 6.51 (d, 1H), 4.09 (q, 1H), 3.92 (m, 1H), 3.84 (dd, 1H), 3.77 (s, 1H), 3.50 (d, 1H), 3.13 (dd, 1H), 3.05 (dd, 1H), 2.96 (dd, 1H), 2.73 (m, 1H), 2.53 (dd, 1H), 2.50-2.40 (m, 4H), 2.40-2.37 (m, 2H), 2.22 (dt, 1H) 1.98 (m, 2H), 1.84 (m, 1H), 1.57 (t, 1H), 1.33 (m, 5H), 0.94-0.84 (m, 8H), 0.84-075 (dd, 1H), 0.73-0.68 (m, 3H). The hydrochloride salt synthesized by the general procedure had mp 210-215° C. dec. [α]_{D}²⁵+75.7° (c 1, MeOH). ¹H NMR (CD₃OD) δ 7.29 (t, 1H, J=9 Hz), 7.12 (d, 1H, J=9 Hz), 6.91 (d, 1H), 6.87-6.61 (m, 3H), 4.48 (d, 1H), 4.35 (d, 1H), 4.30 (m, 1H), 3.81 (s, 3H), 3.78 (d, 1H), 3.63-3.15 (m, 6H), 3.09 (s, 3H), 3.07 (m, 1H), 2.80 (dt, 1H), 2.45 (m, 1H), 1.91 (m, 2H), 1.49 (s, 3H), 1.08-0.90 (m, 7H), 0.86 (d, 3H, J=9 Hz). ESIMS: m/z 494 (M+1, 100). Anal. (C₃₀H₄₅Cl₂N₃O₃H₂O) C, H, N.

### (3R)-7-Methoxy-N-[(1S)-1-{[(3R,4R)-4-(3-methoxyphenyl)-3,4-dimethylpiperidin-1-yl]methyl}-2-methylpropyl]-3-methyl-1,2,3,4-tetrahydroisoquinoline-3-carboxamide (81) Dihydrochloride

General procedure (a) was employed using 126 mg (0.414 mmol) of 7b 140 mg (0.435 mmol) of 6c to afford 51 mg (23%) of the freebase after separation by preparative TLC eluting with 2:1 CHCl₃/CMA-80. ¹H NMR (CD₃OD) δ 7.39-7.19 (m, 2H), 7.92-6.78 (m, 5H), 4.5-4.32 (q, 2H), 4.25 (m, 1H), 3.70 (d, 6H), 3.6-3.30 (m), 3.20 (d, 1H), 2.67 (dt, 1H), 2.45 (m, 1H), 1.92 (bd, 1H), 1.78 (s, 3H), 1.75-1.60 (m, 1H), 1.47 (s, 3H), 0.86 (d, 3H), 0.70 (t, 6H). The hydrochloride salt synthesized by the general procedure had mp>220° C. dec. [α]_{D}²⁵+49.8° (c 1, MeOH). ¹H NMR (CD₃OD) δ 7.30 (t, 1H), 7.26 (d, 1H, J=6 Hz), 6.92-6.80 (m, 2H), 6.84-6.80 (m, 2H), 4.48 (d, 1H), 4.36 (d, 1H), 4.38 (m, 1H), 3.81 (s, 3H), 3.79 (s, 3H), 3.58 (d, 1H), 3.44-3.25 (m, 6H), 3.23 (d, 1H), 2.64 (dt, 1H), 2.46 (m, 1H), 1.95 (d, 1H), 1.78 (s, 3H), 1.80 (m, 1H), 0.83 (d, 3H, J=7.5 Hz), 0.79 (m, 6H). ESIMS: m/z 508 (M+1, 100). Anal. (C₃₁H₄₇Cl₂N₃O₃H₂O) C, H, N.

### (3R)-7-Methoxy-N-[(1S,2S)-1-{[(3R,4R)-4-(3-hydroxyphenyl)-3,4-dimethylpiperidin-1-yl]}-2-methylbutanyl]-3-methyl-1,2,3,4-tetrahydroisoquinoline-3-carboxamide (8m) Dihydrochloride

General procedure (b) was employed using 65 mg (0.213 mmol) of 7c and 46 mg (0.224 mmol) of 6c to afford 25 mg (25%) of the freebase after separation by preparative TLC eluting with 1:1 CMA-80/CH₂Cl₂. ¹H NMR (CDCl₃) δ 7.41 (d, 1H), 7.12 (t, 1H), 6.96 (d, 1H), 6.82-6.52 (m, 5H), 4.18-3.77 (m, 4H), 3.73 (s, 3H), 3.11 (d, 1H), 2.82-2.57 (m, 4H), 2.48-2.28 (m, 4H), 2.17-2.02 (m, 2H), 1.91-1.54 (m, 3H), 1.55-1.22 (m, 9H), 0.98-083 (m, 6H), 0.48 (d, 3H). 1.47 (d, 1H), 1.38 (d, 3H), 1.26-1.17 (m, 7H), 0.82 (t, 6H), 0.58 (d, 3H). The hydrochloride salt synthesized by the general procedure had mp>220° C. dec. [α]_{D}²⁵+44.7° (c 0.45, MeOH). ¹H NMR (CD₃OD) δ 7.24 (d, 1H, J=9 Hz), 7.18 (t, 1H, J=9 Hz), 6.92 (m, 1H), 6.80 (s, 1H), 6.76 (m, 1H), 6.68 (m, 1H), 4.48 (d, 1H), 4.38 (d, 1H), 4.30 (m, 1H), 3.79 (s, 3H), 3.70 (d, 1H, J=15.9 Hz), 3.60-3.31 (m, 5H), 3.20 (d, 1H, J=15.9 Hz), 2.67 (dt, 1H), 2.40 (m, 1H), 1.91 (d, 1H), 1.79 (s, 3H), 1.46 (bs, 4H), 1.13 (m, 1H), 0.85 (d, 3H), 0.80-0.69 (m, 6H). ESIMS: m/z 508 (M+1, 100). Anal. (C₃₁H₄₇Cl₂N₃O₃.2H₂O) C, H, N.

### (3R)-7-Hydroxy-N-[(1S,2S)-1-{[(3R,4R)-4-(3-methoxyphenyl)-3,4-dimethylpiperidin-1-yl]methyl}-2-methylbutanyl)-3-methyl-1,2,3,4-tetrahydroisoquinoline-3-carboxamide (8n) Dihydrochloride

General procedure (a) was employed using 145 mg (0.455 mmol) of 7d and 146 mg (0.478 mmol) of 6a to afford 60 mg (26%) of the freebase after separation by preparative TLC eluting with 3:1 CHCl₃/CMA-80. ¹H NMR (CD₃OD) δ 7.18 (t, 1H, J=8.1 Hz), 6.88 (d, 1H, J=8.1 Hz), 6.82 (d, 1H), 6.78 (t, 1H), 6.89 (dd, 1H, J₂=5.7 Hz, J=2.1 Hz), 6.53 (dd, 1H J₂=5.7 Hz, J=2.1 Hz), 6.46 (d, 1H, J=2.4 Hz), 3.96 (d, 1H), 3.92 (m, 1H), 3.76 (s, 3H), 3.30 (m, 1H), 3.15 (d, 1H, J=15.9 Hz), 2.69 (m, 1H), 2.64 (d, 1H), 2.54 (b, 1H), 2.47-2.36 (m, 5H), 2.17 (dt, 1H), 1.91 (m, 1H), 1.52-1.35 (m, 5H), 1.32 (s, 3H), 1.26 (m, 4H), 1.15 (d, 1H), 1.06-0.9 (m, 2H), 0.86 (t, 3H, J=7.2 Hz), 0.81 (d, 3H, J=6.9 Hz), 0.57 (d, 3H, J=6.9 Hz). The hydrochloride salt synthesized by the general procedure had mp 210° C. dec. [α]_{D}²⁵+39.7° (c 0.41, MeOH). ¹H NMR (CD₃OD) δ 7.27 (t, 1H, J=9 Hz), 7.11 (d, 1H, J=9 Hz), 6.90-6.70 (m, 3H), 6.61 (s, 1H), 4.38 (d, 1H), 4.29 (m, 1H), 3.65 (d, 1H), 3.60-3.25 (m, 5H), 3.15 (d, 1H), 2.64 (dt, 1H), 2.42 (m, 1H), 1.92 (d, 1H), 1.76 (s, 3H), 1.46 (bs, 4H), 1.12 (m, 1H), 0.82 (d, 3H, J=9 Hz), 0.78-0.71 (m, 6H). ESIMS: m/z 508 (M+1, 100). Anal. (C₃₁H₄₇Cl₂N₃O₃.2H₂O) C, H, N.

### (3R)-7-Methoxy-N-[(1S)-1-{[(3R,4R)-4-(3-hydroxyphenyl)-3,4-dimethylpiperidin-1-yl]methyl}-2-methylpropyl]-2,3-dimethyl-1,2,3,4-tetrahydroisoquinoline-3-carboxamide (8o) Dihydrochloride

General procedure (a) was employed using 104 mg (0.358 mmol) of 7a and 88 mg (0.376 mmol) of 6d to afford 80 mg (44%) of the freebase after separation by preparative TLC eluting with 3:1 CHCl₃/CMA-80. ¹H NMR (CD₃OD) δ 7.07 (t, 1H), 6.93 (d, 1H), 6.73-6.52 (m, 5H), 4.86 (s, 3H), 4.07 (d, 1H, J=16.5 Hz), 3.89 (m, 1H), 3.80 (d, 1H, J=16.5 Hz), 3.67 (s, 3H), 3.14 (d, 1H, J=16.5 Hz), 2.72 (m, 1H), 2.62-2.57 (m, 2H), 2.50-2.37 (m, 5H), 2.31 (dd, 1H), 2.18 (dt, 1H), 1.90 (m, 1H), 1.89-1.75 (m, 1H), 1.51 (bd, 1H), 1.37-1.25 (m, 7H), 1.01-0.84 (m, 8H), 0.54 (d, 3H, J=6.9 Hz). The hydrochloride salt synthesized by the general procedure had mp 199° C. dec. [α]_{D}²⁵ +50.2° (c 0.55, MeOH). ¹H NMR (CD₃OD) δ 7.29 (d, 1H, J=9 Hz), 7.18 (t, 1H, J=9 Hz), 6.95 (d, 1H, J=9 Hz), 6.83-6.68 (m, 2H), 6.67 (d, 1H), 4.70-4.48 (bm, 2H), 4.33 (bm, 1H), 3.80 (s, 3H), 3.67-3.30 (m, 7H), 2.99 (s, 1H), 2.68 (dt, 1H), 2.42 (m, 1H), 1.92 (d, 1H), 1.48 (s, 3H), 0.99-0.50 (s, 9H). ESIMS: m/z 508 (M+1, 100). Anal. (C₃₁H₄₇Cl₂N₃O₃.2H₂O) C, H, N.

### (3R)-7-Hydroxy-N-[(1S,2S)-1-{[(3R,4R)-4-(3-methoxyphenyl)-3,4-dimethylpiperidin-1-yl]methyl}-2-methylbutanyl]-2-methyl-1,2,3,4-tetrahydroisoquinoline-3-carboxamide (8p) Dihydrochloride

General procedure (a) was employed using 100 mg (0.328 mmol) of 7d and 71 mg (0.345 mmol) of 6e to afford 40 mg (34%) of the freebase after separation by preparative TLC eluting with 1:1 CMA-80/Et₂O. ¹H NMR (CD₃OD) δ 7.19 (t, 1H, J=7.8 Hz), 6.92-6.84 (m, 2H), 6.82 (s, 1H), 6.71 (d, 1H), 6.59 (m, 1H), 6.21 (m, 1H), 4.09 (q, 1H), 3.98 (m, 1H), 3.87 (m, 1H), 3.77 (s, 3H), 3.49 (d, 1H, J=15 Hz), 3.14-2.82 (m, 4H), 2.79-2.60 (m, 2H), 2.60-2.28 (m, 9H), 2.23 (dt, 1H) 1.97 (m, 1H), 1.68-1.35 (m, 4H), 1.29 (d, 3H), 1.23 (t, 3H), 0.93 (t, 5H), 0.90-0.77 (m, 2H), 0.70 (t, 3H). The hydrochloride salt, synthesized by the general procedure, had mp 180° C. dec. [α]_{D}²⁵ +66.2° (c 0.5, MeOH). ¹H NMR (CD₃OD) δ 7.29 (t, 1H, J=9 Hz), 7.13 (d, 1H, J=9 Hz), 6.93-6.78 (m, 3H), 6.67 (d, 1H), 6.85-6.74 (m, 3H), 6.67 (d, 1H), 4.48-4.28 (m, 3H), 3.81 (s, 3H), 3.63 (d, 1H), 3.60-3.25 (m, 6H), 3.09-3.01 (m, 4H), 2.78 (dt, 1H), 2.46 (m, 1H), 1.92 (m, 1H), 1.78 (s, 3H), 1.48 (bs, 4H), 1.1-0.8 (m, 10H). ESIMS: m/z 508 (M+1, 100). Anal. (C₃₁H₄₇Cl₂N₃O₃.H₂O) C, H, N.

Abbreviations: GPCRs, G-protein-coupled receptors; cDNAs, complementary deoxyribonucleic acid; SAR, structure activity relationship; [³⁵S]GTPγS, sulfur-35 guanosine-5′-O-(3-thio)triphosphate; DAMGO, [D-Ala²,MePhe⁴,Gly-ol⁵]enkephalin; DPDPE, [D-Pen²,D-Pen⁵]enkephalin; U69,593, (5α,7α,8β)-(−)-N-methyl-N-[7-(1-pyrrolidinyl)-1-oxaspiro[4,5]dec-8-yl]benzeneacetamide; CHO, Chinese hamster ovary; GDP, guanosine diphosphate; BOP, benzotriazole-1-yloxy-tris(dimethylamino)phosphonium hexafluorophosphate; HBTU, O-(benzotriazol-1-yl)-N,N,N′,N′-tetramethyluronium hexafluorophosphate; Tic, tetrahydroisoquinolinecarboxylic acid; tPSA, topological polar surface area.

TABLE 1
Comparison of Inhibition of Agonist Stimulated [³⁵S]GTPγS
Binding in Cloned Human μ, δ, and κ-opioid Receptors ^{a}

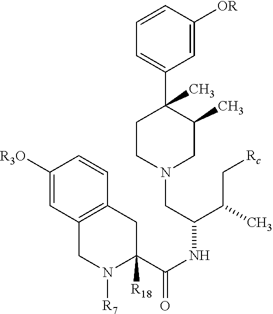

| | | | | | | μ, DAMGO | δ, DPDPE | κ, U69,593 | | |
|---|---|---|---|---|---|---|---|---|---|---|
| Compound | Rₐ | R | R_{c} | R₁₈ | R₇ | Kₑ (nM) | Kₑ (nM) | Kₑ (nM) | μ/κ | δ/κ |
| 1 | | | | | | 26 ± 7 | 29 ± 8 | 0.05 ± 0.02 | 520 | 580 |
| 3 | H | H | H | H | H | 25.1 ± 3.5^{b} | 76.4 ± 2.7^{b} | 0.02 ± 0.01^{b} | 1255 | 3830 |
| 8a | H | H | CH₃ | H | H | 3 ± 1^{c} | 24 ± 4^{c} | 0.03 ± 0.02^{c} | 100 | 800 |
| 8b | CH₃ | H | H | H | H | 51.4 ± 15^{c} | 118 ± 45^{c} | 0.06 ± 0.01^{c} | 857 | 1970 |
| 8c | H | H | H | H | CH₃ | 210 ± 60 | 491 ± 120 | 0.16 ± 0.06 | 1313 | 3070 |
| 8d | H | CH₃ | H | H | H | 24 ± 8 | 21.2 ± 5 | 0.037 ± 0.003 | 649 | 573 |
| 8e | H | H | H | CH₃ | H | 3.6 ± 1 | 854 ± 210 | 0.03 ± 0.008 | 120 | 28500 |
| 8f | H | CH₃ | CH₃ | H | H | 5.1 ± 2 | 1170 ± 400 | 0.96 ± 0.4 | 5 | 1220 |
| 8g | CH₃ | H | H | CH₃ | H | 3.8 ± 1.1 | 36.8 ± 6.9 | 0.93 ± 0.05 | 4 | 40 |
| 8h | H | CH₃ | H | CH₃ | H | 123 ± 30 | 2200 ± 900 | 0.26 ± 0.1 | 473 | 8500 |
| 8i | H | H | CH₃ | CH₃ | H | 8.7 ± 2.7 | 149 ± 13 | 0.11 ± 0.01 | 79 | 1350 |
| 8j | H | H | CH₃ | H | CH₃ | 7.2 ± 1.8 | 132 ± 24 | 0.11 ± 0.03 | 66 | 1200 |
| 8k | H | CH₃ | H | H | CH₃ | 880 ± 220 | 2300 ± 900 | 4.3 ± 2.7 | 204 | 535 |
| 8l | CH₃ | CH₃ | H | CH₃ | H | 1450 ± 490 | IA^{c} | 15.2 ± 3.7 | 95 | — |
| 8m | CH₃ | H | CH₃ | CH₃ | H | 17.5 ± 3.6 | 18.7 ± 1.5 | 3.5 ± 0.8 | 5 | 5 |
| 8n | H | CH₃ | CH₃ | CH₃ | H | 59.1 ± 16 | 2100 ± 600 | 0.52 ± 0.2 | 114 | 4040 |
| 8o | CH₃ | H | H | CH₃ | CH₃ | 7.0 ± 1.4 | 117 ± 30 | 2.2 ± 0.6 | 3 | 53 |
| 8p | H | CH₃ | CH₃ | H | CH₃ | 360 ± 120 | IA^{d} | 2.03 ± 0.03 | 180 | — |

^{a} The data represent the means ± SE from at least three independent experiments.
^{b} The Kₑ values for 3 supplied by the NIDA Opioid Treatment Discovery Program (OTDP) were 3.41, 79.3, and 0.01 nM for the μ, δ, and κ receptors, respectively (ref. 14).
^{c} Inactive or >10,000 nM.

TABLE 2
Calculated logP, tPSA, and logBB^{a}

| Compd | logP | tPSA | logBB |
|---|---|---|---|
| 1 | 1.57 | 121.65 | −1.42 |
| 3 | 3.75 | 84.83 | −0.55 |
| 8a | 4.09 | 84.83 | −0.49 |
| 8b | 4.11 | 73.83 | −0.33 |
| 8c | 4.12 | 76.04 | −0.36 |
| 8d | 3.83 | 73.83 | −0.37 |
| 8e | 3.98 | 84.83 | −0.51 |
| 8f | 4.18 | 73.83 | −0.32 |
| 8g | 4.33 | 73.83 | −0.30 |
| 8h | 4.06 | 73.83 | −0.34 |
| 8i | 4.32 | 84.83 | −0.46 |
| 8j | 4.46 | 76.04 | −0.31 |
| 8k | 4.20 | 65.04 | −0.19 |
| 8l | 4.74 | 62.83 | −0.07 |
| 8m | 4.71 | 73.83 | −0.24 |
| 8n | 4.41 | 73.83 | −0.28 |
| 8o | 4.69 | 65.04 | −0.11 |
| 8p | 4.55 | 65.04 | −0.13 |

^{a}logBB was calculated using equation 6 in reference 24.

Obviously, numerous modifications and variations of the present invention are possible in light of the above teachings. It is therefore to be understood that within the scope of the appended claims, the invention may be practiced otherwise than as specifically described herein.

### REFERENCES

(1) Volkow, N. D.; Li, T. K. Drug addiction: the neurobiology of behaviour gone awry. Nat. Rev. Neurosci. 2004, 5, 963-970.
(2) Koob, G.; Kreek, M. J. Stress, dysregulation of drug reward pathways, and the transition to drug dependence. Am. J. Psychiatry 2007, 164, 1149-1159.
(3) Nestler, E. J.; Carlezon, W. A., Jr. The mesolimbic dopamine reward circuit in depression. Biol. Psychiatry 2006, 59, 1151-1159.
(4) Land, B. B.; Bruchas, M. R.; Lemos, J. C.; Xu, M.; Melief, E. J.; Chavkin, C. The dysphoric component of stress is encoded by activation of the dynorphin kappa-opioid system. J. Neurosci. 2008, 28, 407-414.
(5) Pfeiffer, A.; Brantl, V.; Herz, A.; Emrich, H. M. Psychotomimesis mediated by kappa opiate receptors. Science 1986, 233, 774-776.
(6) Carlezon, W. A., Jr.; Beguin, C.; DiNieri, J. A.; Baumann, M. H.; Richards, M. R.; Todtenkopf, M. S.; Rothman, R. B.; Ma, Z.; Lee, D. Y.; Cohen, B. M. Depressive-like effects of the kappa-opioid receptor agonist salvinorin A on behavior and neurochemistry in rats. J. Pharmacol. Exp. Ther. 2006, 316, 440-447.
(7) Carey, A. N.; Borozny, K.; Aldrich, J. V.; McLaughlin, J. P. Reinstatement of cocaine place-conditioning prevented by the peptide kappa-opioid receptor antagonist arodyn. Eur. J. Pharmacol. 2007, 569, 84-89.
(8) Beardsley, P. M.; Howard, J. L.; Shelton, K. L.; Carroll, F. I. Differential effects of the novel kappa opioid receptor antagonist, JDTic, on reinstatement of cocaine-seeking induced by footshock stressors vs cocaine primes and its antidepressant-like effects in rats. Psychopharmacology (Berl) 2005, 183, 118-126.
(9) McLaughlin, J. P.; Marton-Popovici, M.; Chavkin, C. Kappa opioid receptor antagonism and prodynorphin gene disruption block stress-induced behavioral responses. J. Neurosci. 2003, 23, 5674-5683.
(10) Mague, S. D.; Pliakas, A. M.; Todtenkopf, M. S.; Tomasiewicz, H. C.; Zhang, Y.; Stevens, W. C., Jr.; Jones, R. M.; Portoghese, P. S.; Carlezon, W. A., Jr. Antidepressant-like effects of kappa-opioid receptor antagonists in the forced swim test in rats. J. Pharmacol. Exp. Ther. 2003, 305, 323-330.
(11) Portoghese, P. S.; Nagase, H.; Lipkowski, A. W.; Larson, D. L.; Takemori, A. E. Binaltorphimine-related bivalent ligands and their kappa opioid receptor antagonist selectivity [published erratum appears in J. Med. Chem. 1988 October; 31(10):2056]. J. Med. Chem. 1988, 31, 836-841.
(12) Jones, R. M.; Hjorth, S. A.; Schwartz, T. W.; Portoghese, P. S. Mutational evidence for a common kappa antagonist binding pocket in the wild-type kappa and mutant mu[K303E] opioid receptors. J. Med. Chem. 1998, 41, 4911-4914.
(13) Thomas, J. B.; Atkinson, R. N.; Rothman, R. B.; Fix, S. E.; Mascarella, S. W.; Vinson, N. A.; Xu, H.; Dersch, C. M.; Lu, Y.; Cantrell, B. E.; Zimmerman, D. M.; Carroll, F. I. Identification of the first trans-(3R,4R)-dimethyl-4-(3-hydroxyphenyl)piperidine derivative to possess highly potent and selective opioid kappa receptor antagonist activity. J. Med. Chem. 2001, 44, 2687-2690.
(14) Thomas, J. B.; Atkinson, R. N.; Vinson, N. A.; Catanzaro, J. L.; Perretta, C. L.; Fix, S. E.; Mascarella, S. W.; Rothman, R. B.; Xu, H.; Dersch, C. M.; Cantrell, B. E.; Zimmerman, D. M.; Carroll, F. I. Identification of (3R)-7-hydroxy-N-((1S)-1-[[(3R,4R)-4-(3-hydroxyphenyl)-3,4-dimethyl-1-piperidinyl]methyl]-2-methylpropyl)-1,2,3,4-tetrahydro-3-isoquinolinecarboxamide as a novel potent and selective opioid kappa receptor antagonist. J. Med. Chem. 2003, 46, 3127-3137.
(15) Bennett, M. A.; Murray, T. F.; Aldrich, J. V. Identification of arodyn, a novel acetylated dynorphin A-(1-11) analogue, as a kappa opioid receptor antagonist. J. Med. Chem. 2002, 45, 5617-5619.
(16) Metcalf, M. D.; Coop, A. Kappa opioid antagonists: past successes and future prospects. AAPS J. 2005, 7, E704-E722.
(17) Knoll, A. T.; Meloni, E. G.; Thomas, J. B.; Carroll, F. I.; Carlezon, W. A., Jr. Anxiolytic-Like Effects of κ-Opioid Receptor Antagonists in Models of Unlearned and Learned Fear in Rats. J. Pharmacol. Exp. Ther. 2007, 323, 838-845.
(18) Redila, V. A.; Chavkin, C. Stress-induced reinstatement of cocaine seeking is mediated by the kappa opioid system. Psychopharmacology (Berl) 2008, 200, 59-70.
(19) Walker, B. M.; Koob, G. F. Pharmacological evidence for a motivational role of 1c-opioid systems in ethanol dependence. Neuropsychopharmacology 2007, 1-10.
(20) Bodnar, R. J.; Glass, M. J.; Ragnauth, A.; Cooper, M. L. General, mu and kappa opioid antagonists in the nucleus accumbens alter food intake under deprivation, glucoprivic and palatable conditions. Brain Res. 1995, 700, 205-212.
(21) Bortolato, M.; Aru, G. N.; Frau, R.; Orru, M.; Fa, M.; Manunta, M.; Puddu, M.; Mereu, G.; Gessa, G. L. Kappa opioid receptor activation disrupts prepulse inhibition of the acoustic startle in rats. Biol. Psychiatry 2005, 57, 1550-1558.
(22) Carroll, F. I.; Thomas, J. B.; Dykstra, L. A.; Granger, A. L.; Allen, R. M.; Howard, J. L.; Pollard, G. T.; Aceto, M. D.; Harris, L. S. Pharmacological properties of JDTic: A novel κ-opioid receptor antagonist. Eur. J. Pharmacol. 2004, 501, 111-119.
(23) Cai, T. B.; Zou, Z.; Thomas, J. B.; Brieaddy, L.; Navarro, H. A.; Carroll, F. I. Synthesis and in vitro opioid receptor functional antagonism of analogues of the selective kappa opioid receptor antagonist (3R)-7-hydroxy-N-((1S)-1-{[(3R,4R)-4-(3-hydroxyphenyl)-3,4-dimethyl-1-piperidinyl]methyl}-2-methylpropyl)-1,2,3,4-tetrahydro-3-isoquinolinecarboxamide (JDTic). J. Med. Chem. 2008, 51, 1849-1860.
(24) Clark, D. E. Rapid calculation of polar molecular surface area and its application to the prediction of transport phenomena. 2. Prediction of blood-brain barrier penetration. J. Pharm. Sci. 1999, 88, 815-821.
(25) Hsiao, Y.; Hegedus, L. S. Synthesis of optically active imidazolines, azapenams, dioxocyclams, and bis-dioxocyclams. J. Org. Chem. 1997, 62, 3586-3591.
(26) Ma, D.; Ma, Z.; Kozikowski, A. P.; Pshenichkin, S.; Wroblewski, J. T. Synthesis and biological evaluation of two analogues of (S)-alpha-methyl-3-carboxyphenylalanine. Bioorg. Med. Chem. Lett. 1998, 8, 2447-2450.
(27) Zimmerman, D. M.; Leander, J. D.; Cantrell, B. E.; Reel, J. K.; Snoddy, J.; Mendelsohn, L. G.; Johnson, B. G.; Mitch, C. H. Structure-activity relationships of the trans-3,4-dimethyl-4-(3-hydroxyphenyl)piperidine antagonists for μ and κ opioid receptors. J. Med. Chem. 1993, 36, 2833-2841.
(28) Thomas, J. B.; Fall, M. J.; Cooper, J. B.; Rothman, R. B.; Mascarella, S. W.; Xu, H.; Partilla, J. S.; Dersch, C. M.; McCullough, K. B.; Cantrell, B. E.; Zimmerman, D. M.; Carroll, F. I. Identification of an opioid K receptor subtype-selective N-substituent for (+)-(3R,4R)-dimethyl-4-(3-hydroxyphenyl)piperidine. J. Med. Chem. 1998, 41, 5188-5197.
(29) Carroll, F. I.; Chaudhari, S.; Thomas, J. B.; Mascarella, S. W.; Gigstad, K. M.; Deschamps, J.; Navarro, H. A. N-Substituted cis-4-a-(3-hydroxyphenyl)-8a-methyloctahydroisoquinolines are opioid receptor pure antagonists. J. Med. Chem. 2005, 48, 8182-8193.
(30) Liu, C.; Thomas, J. B.; Brieaddy, L.; Berrang, B.; Carroll, F. I. An improved synthesis of (3R)-2-(tert-butoxycarbonyl-D-7-hydroxy-1,2,3,4-tetrahydroisoquinoline-3-carboxylic Acid. Synthesis 2008, 856-858.
(31) U.S. Patent Publication No. 2002/0132828 and U.S. Pat. No. 6,974,824.
(32) U.S. Patent Publication No. 2006/0183743.
(33) U.S. Patent Publication No. 2009/0264462.

1. A kappa opioid receptor antagonist represented by the formula (I): 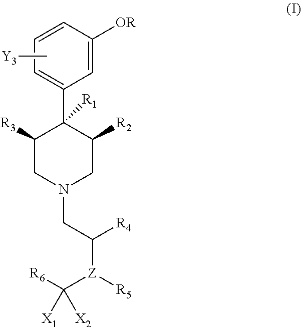
wherein
R is C₁₋₈ alkyl, C₁₋₈ haloalkyl, C₃₋₈ alkenyl, C₃₋₈ alkynyl, aryl substituted by one or more groups Y₁ or CH₂-aryl substituted by one or more groups Y₁;
R₁ is one of the following structures: 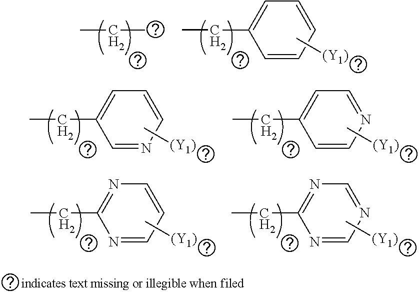
each Y₁ is, independently, hydrogen, OH, Br, Cl, F, CN, CF₃, NO₂, N₃, OR₈, CO₂R₉, C₁₋₆ alkyl, NR₁₀R₁₁, NHCOR₁₂, NHCO₂R₁₂, CONR₁₃R₁₄, or CH₂(CH₂)ₙY₂;
Y₂ is hydrogen, CF₃, CO₂R₉, C₁₋₆ alkyl, NR₁₀R₁₁, NHCOR₁₂, NHCO₂R₁₂, CONR₁₃R₁₄, CH₂OH, CH₂OR₈, or COCH₂R₉;
Y₃ is hydrogen, OH, Br, Cl, F, CN, CF₃, NO₂, N₃, OR₈, CO₂R₉, C₁₋₆ alkyl, NR₁₀R₁₁, NHCOR₁₂, NHCO₂R₁₂, CONR₁₃R₁₄, or CH₂(CH₂)ₙY₂;
R₂ is hydrogen, C₁₋₈ alkyl, C₃₋₈ alkenyl, C₃₋₈ alkynyl or CH₂-aryl substituted by one or more groups Y₁;
R₃ is hydrogen, C₁₋₈ alkyl, C₃₋₈ alkenyl, C₃₋₈ alkynyl or CH₂-aryl substituted by one or more groups Y₁,
wherein R₂ and R₃ may be bonded together to form a C₂₋₈ alkyl group;
R₄ is hydrogen, C_{1-g} alkyl, CO₂C₁₋₈ alkylaryl substituted by one or more groups Y₁, CH₂-aryl substituted by one or more groups Y₁ or CO₂C₁₋₈ alkyl;
Z is N, O or S, wherein when Z is O or S, there is no R₅;
R₅ is hydrogen, C₁₋₈ alkyl, C₃₋₈ alkenyl, C₃₋₈ alkynyl, CH₂CO₂C₁₋₈ alkyl, CO₂C₁₋₈ alkyl or CH₂-aryl substituted by one or more groups Y₁;
n is 0, 1, 2 or 3;
o is 0, 1, 2 or 3;
R₆ is a group selected from the group consisting of structures (a)-(p): 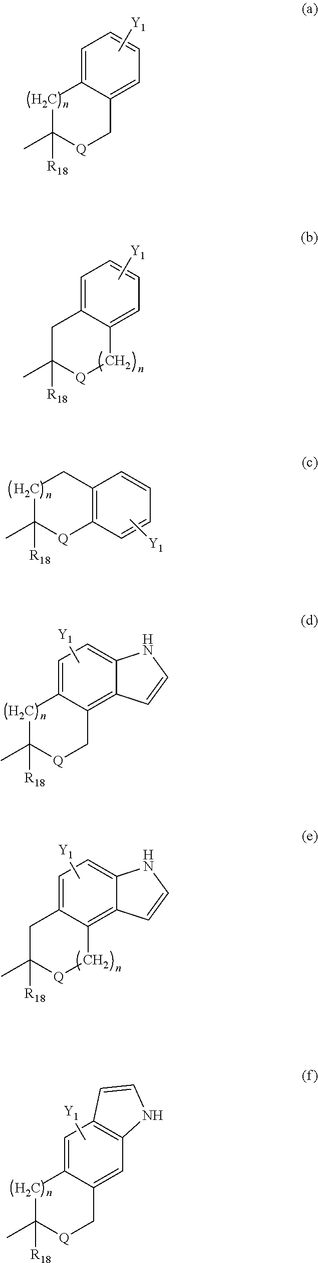 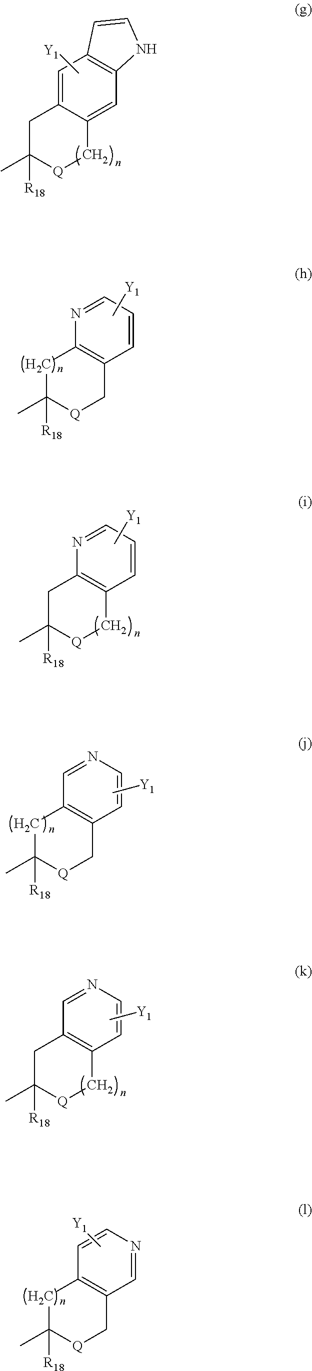 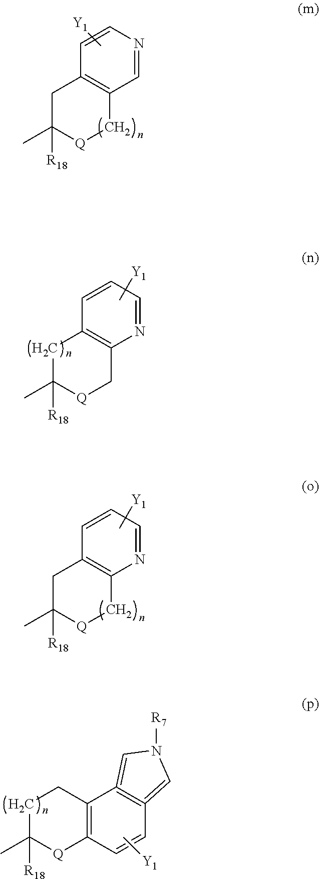
Q is NR₇, CH₂, O, S, SO, or SO₂;
X₁ is hydrogen, C₁₋₈ alkyl, C₃₋₈ alkenyl, or C₃₋₈ alkynyl;
X₂ is hydrogen, C₁₋₈alkyl, alkenyl, or C₃₋₈ alkynyl;
or X₁ and X₂ together form ═O, ═S, or ═NH;
each R₇ is, independently, H, C₁₋₈ alkyl, CH₂-aryl substituted by one or more substituents Y₁, NR₁₀R₁₁, NHCOR₁₂, NHCO₂R₁₃, CONR₁₄R₁₅, CH₂(CH₂)ₙY₂, or C(═NH)NR₁₆R₁₇,
each of R₈, R₉, R₁₀, R₁₁, R₁₂, R₁₃, R₁₄, R₁₅, R₁₆ and R₁₇ is, independently, hydrogen, C₁₋₈ alkyl, CH₂-aryl substituted by one or more substituents OH, Br, Cl, F, CN, CF₃, NO₂, N₃, C₁₋₆ alkyl, or CH₂(CH₂)Y₂′;
Y₂′ is hydrogen, CF₃, or C₁₋₆ alkyl;
R₁₈ is hydrogen, C₁₋₈ alkyl, C₂₋₈ alkenyl, C₃₋₈ alkynyl, or CH₂-aryl substituted by one or more groups Y₁;
or a pharmaceutically acceptable salt thereof.

2. The kappa opioid receptor antagonist of claim 1, wherein
R is C₁₋₈ alkyl or C₁₋₈ haloalkyl, phenyl substituted by one or more groups Y₁ or CH₂-phenyl substituted by one or more groups Y₁;
R₁ is C₁₋₃ alkyl;
Y₃ is hydrogen or C₁₋₆ alkyl;
R₂ is hydrogen or C₁₋₈ alkyl,
R₃ is hydrogen or C₁₋₈ alkyl,
or R₂ and R₃ are bonded together to form a C₂₋₈ alkyl group
R₄ is hydrogen or C₁₋₈ alkyl;
Z is N;
X₁ and X₂ together form ═O;
R₆ is represented by the formula (a), (b) or (c); and
R₁₈ is hydrogen or C₁₋₈ alkyl,
or a pharmaceutically acceptable salt thereof.

3. The kappa opioid receptor antagonist of claim 1, wherein
R is C₁₋₄ alkyl or C₁₋₄ haloalkyl;
R₁ is C₁₋₃ alkyl;
Y₃ is hydrogen or C₁₋₄ alkyl;
R₂ is hydrogen or C₁₋₄ alkyl,
R₃ is hydrogen or C₁₋₄ alkyl;
or R₂ and R₃ are bonded together to form a C₂₋₈ alkyl group
R₄ is hydrogen or C₁₋₆ alkyl;
Z is N;
X₁ and X₂ together form ═O;
R₆ is represented by the formula (a), (b) or (c); and
R₁₈ is hydrogen or C₁₋₄ alkyl,
or a pharmaceutically acceptable salt thereof.

4. The kappa opioid receptor antagonist of claim 1, wherein
R is C₁₋₂ alkyl or C₁₋₂ haloalkyl;
R₁ is C₁₋₂ alkyl;
Y₃ is hydrogen or C₁₋₂ alkyl;
R₂ is hydrogen or C₁₋₂ alkyl,
R₃ is hydrogen or C₁₋₂ alkyl,
or R₂ and R₃ are bonded together to form a C₂₋₈ alkyl group;
R₄ is hydrogen or C₁₋₆ alkyl;
Z is N;
X₁ and X₂ together form ═O;
R₆ is represented by the formula (a), (b) or (c); and
R₁₈ is hydrogen or C₁₋₂ alkyl,
or a pharmaceutically acceptable salt thereof.

5. The kappa opioid receptor antagonist of claim 1, wherein
R is methyl or trifluoromethyl;
R₁ is methyl;
Y₃ is hydrogen or methyl;
R₂ is hydrogen or methyl,
R₃ is hydrogen or methyl;
R₄ is hydrogen or C₁₋₆ alkyl;
Z is N;
X₁ and X₂ together form ═O;
R₆ is represented by the formula (a), (b) or (c); and
R₁₈ is hydrogen or methyl,
or a pharmaceutically acceptable salt thereof.

6. The kappa opioid receptor antagonist of claim 1, wherein
R is methyl or trifluoromethyl;
R₁ is methyl;
Y₃ is hydrogen;
R₂ is methyl,
R₃ is hydrogen;
R₄ is hydrogen or C₁₋₄ alkyl;
Z is N;
X₁ and X₂ together form ═O;
R₆ is represented by the formula (a), (b) or (c);
R₁₈ is hydrogen or C₁₋₂ alkyl,
Q is NR₇;
R₇ is hydrogen or C₁₋₈ alkyl;
Y₁ is hydrogen, OH or OR₈;
R₈ is C₁₋₈ alkyl; and
n is 0, 1 or 2,
or a pharmaceutically acceptable salt thereof.

7. The kappa opioid receptor antagonist of claim 1, wherein
R is methyl or trifluoromethyl;
R₁ is methyl;
Y₃ is hydrogen;
R₂ is methyl,
R₃ is hydrogen;
R₄ is hydrogen or C₁₋₄ alkyl;
Z is N;
X₁ and X₂ together form ═O;
R₆ is represented by the formula (a), (b) or (c);
R₁₈ is hydrogen or methyl,
Q is NR₇;
R₇ is hydrogen or C₁₋₄ alkyl;
Y₁ is hydrogen, OH or OR₈;
R₈ is C₁₋₄ alkyl; and
n is 0 or 1,
or a pharmaceutically acceptable salt thereof.

8. The kappa opioid receptor antagonist of claim 1, wherein
R is methyl or trifluoromethyl;
R₁ is methyl;
Y₃ is hydrogen;
R₂ is methyl,
R₃ is hydrogen;
R₄ is hydrogen or C₁₋₄ alkyl;
Z is N;
X₁ and X₂ together form ═O;
R₆ is represented by the formula (a), (b) or (c);
R₁₈ is hydrogen or methyl,
Q is NR₇;
R₇ is hydrogen or C₁₋₂ alkyl;
Y₁ is hydrogen, OH or OR₈;
R₈ is C₁₋₂ alkyl; and
n is 0 or 1,
or a pharmaceutically acceptable salt thereof.

9. The kappa opioid receptor antagonist of claim 1, wherein
R is methyl;
R₁ is methyl;
Y₃ is hydrogen;
R₂ is methyl,
R₃ is hydrogen;
R₄ is hydrogen or C₁₋₄ alkyl;
Z is N;
X₁ and X₂ together form ═O;
R₆ is represented by the formula (a), (b) or (c);
R₁₈ is hydrogen or methyl,
Q is NR₇;
R₇ is hydrogen or methyl;
Y₁ is OH or OR₈;
R₈ is methyl; and
n is 0 or 1,
or a pharmaceutically acceptable salt thereof.

10. The kappa opioid receptor antagonist of claim 1, wherein
R is trifluoromethyl;
R₁ is methyl;
Y₃ is hydrogen;
R₂ is methyl,
R₃ is hydrogen;
R₄ is hydrogen or C₁₋₄ alkyl;
Z is N;
X₁ and X₂ together form ═O;
R₆ is represented by the formula (a), (b) or (c);
R₁₈ is hydrogen or methyl,
Q is NR₇;
R₇ is hydrogen or methyl;
Y₁ is OH or OR₈;
R₈ is methyl; and
n is 0 or 1,
or a pharmaceutically acceptable salt thereof.

11. The kappa opioid receptor antagonist of claim 1, which is represented by the formula: 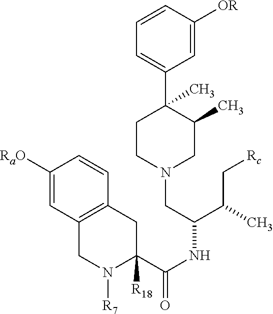
wherein
(1) Rₐ is hydrogen;
R is methyl;
R_{c} is hydrogen;
R₇ is hydrogen; and
R₁₈ is hydrogen,
or
(2) Rₐ is hydrogen;
R is methyl;
R_{c} is methyl;
R₇ is hydrogen; and
R₁₈ is hydrogen,
or
(3) Rₐ is hydrogen;
R is methyl;
R_{c} is hydrogen;
R₇ is hydrogen; and
R₁₈ is methyl,
or
(4) Rₐ is hydrogen;
R is methyl;
R_{c} is hydrogen
R₇ is methyl, and
R₁₈ is hydrogen,
or
(5) Rₐ is methyl;
R is methyl;
R_{c} is hydrogen;
R₇ is hydrogen; and
R₁₈ is methyl,
or
(6) Rₐ is hydrogen;
R is methyl;
R_{c} is methyl;
R₇ is hydrogen; and
R₁₈ is methyl,
or
(7) Rₐ is hydrogen;
R is methyl;
R_{c} is methyl;
R₇ is methyl;
R₁₈ is hydrogen,
or a pharmaceutically acceptable salt thereof.

12. The kappa opioid receptor antagonist of claim 1, which has a κ/μ selectivity of at least 2:1.

13. The kappa opioid receptor antagonist of claim 1, which has a κ/μ selectivity of at least 50:1.

14. The kappa opioid receptor antagonist of claim 1, which has a κ/μ selectivity of at least 100:1.

15. The kappa opioid receptor antagonist of claim 1, which has a κ/δ selectivity of at least 2:1.

16. The kappa opioid receptor antagonist of claim 1, which has a κ/δ selectivity of at least 20,000:1.

17. The kappa opioid receptor antagonist of claim 1, which has a κ/δ selectivity of at least 25,000:1.

18. The kappa opioid receptor antagonist of claim 1, which has a κ/μ selectivity of at least 100:1 and which has a κ/δ selectivity of at least 20,000:1.

19. A pharmaceutical composition comprising:
an effective amount of the kappa opioid receptor antagonist of claim 1 or a pharmaceutically acceptable salt thereof and a physiologically acceptable carrier.

20. The pharmaceutical composition of claim 19, which is an injectable composition.

21. The pharmaceutical composition of claim 19, which is an orally administrable composition.

22. The pharmaceutical composition of claim 19, which is an orally administrable composition in a form selected from the group consisting of tablets, capsules, troches, powders, solutions, dispersions, emulsions and suspensions.

23. A method of making the pharmaceutical composition of claim 19, comprising combining the kappa opioid receptor antagonist or a pharmaceutically acceptable salt thereof and the physiologically acceptable carrier.

24. A method of binding a kappa opioid receptor in a subject in need thereof, comprising administering to the subject an effective amount of the kappa opioid receptor antagonist of claim 1 or a pharmaceutically acceptable salt thereof.

25. A method of treating substance abuse, comprising administering a subject in need thereof an effective amount of the kappa opioid receptor antagonist of claim 1 or a pharmaceutically acceptable salt thereof.

26. A method of eliminating or suppressing withdrawal from an addictive substance, comprising administering a subject in need thereof an effective amount of the kappa opioid receptor antagonist of claim 1 or a pharmaceutically acceptable salt thereof.

27. A method of treating a subject having at least one disease state that is ameliorated by binding an opioid receptor and/or temporary suppression of the kappa opioid receptor system, comprising administering a subject in need thereof an effective amount of the kappa opioid receptor antagonist of claim 1 or a pharmaceutically acceptable salt thereof.

28. A method of treating one or more conditions selected from the group consisting of migraines, arthritis, allergy, viral infections, diarrhea, psychosis, schizophrenia, depression, uropathy, addiction, obesity, comprising administering a subject in need thereof an effective amount of the kappa opioid receptor antagonist of claim 1 or a pharmaceutically acceptable salt thereof.

29. A method of providing one or more of effects selected from the group consisting of cytostatic, immunomodulatory, immunosuppressive, antitussive, hypotensive agents, anti-diuretic, stimulatory and anti-convulsant, comprising administering a subject in need thereof an effective amount of the kappa opioid receptor antagonist of claim 1 or a pharmaceutically acceptable salt thereof.

30. A method treat and/or prevent paralysis resulting from traumatic ischemia and/or neuroprotection against ischemic trauma, comprising administering a subject in need thereof an effective amount of the kappa opioid receptor antagonist of claim 1 or a pharmaceutically acceptable salt thereof.

31. A method of providing an adjunct to nerve growth factor treatment of hyperalgesia and nerve grafts, comprising administering a subject in need thereof an effective amount of the kappa opioid receptor antagonist of claim 1 or a pharmaceutically acceptable salt thereof.

32. A method of treating Parkinson's disease comprising administering to a subject in need thereof the kappa opioid receptor antagonist of claim 1 or a pharmaceutically acceptable salt thereof with L-dopa.

33. A method of treating addiction, comprising administering to a subject in need thereof an effective amount of the kappa opioid receptor antagonist of claim 1 or a pharmaceutically acceptable salt thereof.

34. The method of claim 33, wherein the addiction is to cocaine, alcohol, methamphetamine, nicotine or heroine, a

35. A method of treating nicotine withdrawal effects, comprising administering to a subject in need thereof an effective amount of the kappa opioid receptor antagonist of claim 1 or a pharmaceutically acceptable salt thereof.
